SEQUENCE LISTING (59 sequences)

SEQ ID NO: 1 - protein, 470 aa, Artificial Sequence
  Mouse monoclonal anti-MMP9 antibody - AB0041 heavy chain
MAVLVLFLCLVAFPSCVLSQVQLKESGPGLVAPSQSLSITCTVSGFSLLSYGVHWVRQPPGKGLEWLGVIWTGGTTNYNSALMSRLSISKDDSKSQVFLKMNSLQTDDTAIYYCARYYYGMDYWGQGTSVTVSSAKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVRISWFVNNVEVHTAQTQTHREDYNSTIRVVSALPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLDIKTSKWEKTDSFSCNVRHEGLKNYYLKKTISRSPGK

SEQ ID NO: 2 - protein, 234 aa, Artificial Sequence
  Mouse monoclonal anti-MMP9 antibody - AB0041 light chain
MESQIQVFVFVFLWLSGVDGDIVMTQSHKFMSTSVGDRVSITCKASQDVRNTVAWYQQKTGQSPKLLIYSSSYRNTGVPDRFTGSGSGTDFTFTISSVQAEDLAVYFCQQHYITPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 3 - protein, 115 aa, Artificial Sequence
  Variable region of the IgG2b heavy chain of AB0041
QVQLKESGPGLVAPSQSLSITCTVSGFSLLSYGVHWVRQPPGKGLEWLGVIWTGGTTNYNSALMSRLSISKDDSKSQVFLKMNSLQTDDTAIYYCARYYYGMDYWGQGTSVTVSS

SEQ ID NO: 4 - protein, 107 aa, Artificial Sequence
  Variable region of the kappa light chain of AB0041
DIVMTQSHKFMSTSVGDRVSITCKASQDVRNTVAWYQQKTGQSPKLLIYSSSYRNTGVPDRFTGSGSGTDFTFTISSVQAEDLAVYFCQQHYITPYTFGGGTKLEIK

SEQ ID NO: 5 - protein, 115 aa, Artificial Sequence
  Heavy chain  variant VH1 for AB0041
QVQLQESGPGLVKPSETLSLTCTVSGFSLLSYGVHWVRQPPGKGLEWLGVIWTGGTTNYNSALMSRLTISKDDSKSTVYLKMNSLKTEDTAIYYCARYYYGMDYWGQGTSVTVSS

SEQ ID NO: 6 - protein, 115 aa, Artificial Sequence
  Heavy chain  variant VH2 for AB0041
QVQLQESGPGLVKPSETLSLTCTVSGFSLLSYGVHWVRQPPGKGLEWLGVIWTGGTTNYNSALMSRLTISKDDSKNTVYLKMNSLKTEDTAIYYCARYYYGMDYWGQGTLVTVSS

SEQ ID NO: 7 - protein, 115 aa, Artificial Sequence
  Heavy chain  variant VH3 for AB0041
QVQLQESGPGLVKPSETLSLTCTVSGFSLLSYGVHWVRQPPGKGLEWLGVIWTGGTTNYNSALMSRFTISKDDSKNTVYLKMNSLKTEDTAIYYCARYYYGMDYWGQGTLVTVSS

SEQ ID NO: 8 - protein, 115 aa, Artificial Sequence
  Heavy chain  variant VH4 for AB0041
QVQLQESGPGLVKPSETLSLTCTVSGFSLLSYGVHWVRQPPGKGLEWLGVIWTGGTTNYNSALMSRFTISKDDSKNTLYLKMNSLKTEDTAIYYCARYYYGMDYWGQGTLVTVSS

SEQ ID NO: 9 - protein, 107 aa, Artificial Sequence
  Light chain  variant Vk1 for AB0041
DIVMTQSPSFLSASVGDRVTITCKASQDVRNTVAWYQQKTGKAPKLLIYSSSYRNTGVPDRFTGSGSGTDFTLTISSLQAEDVAVYFCQQHYITPYTFGGGTKVEIK

SEQ ID NO: 10 - protein, 107 aa, Artificial Sequence
  Light chain  variant Vk2 for AB0041
DIVMTQSPSSLSASVGDRVTITCKASQDVRNTVAWYQQKPGKAPKLLIYSSSYRNTGVPDRFTGSGSGTDFTLTISSLQAEDVAVYFCQQHYITPYTFGGGTKVEIK

SEQ ID NO: 11 - protein, 107 aa, Artificial Sequence
  Light chain  variant Vk3 for AB0041
DIQMTQSPSSLSASVGDRVTITCKASQDVRNTVAWYQQKPGKAPKLLIYSSSYRNTGVPDRFSGSGSGTDFTLTISSLQAEDVAVYFCQQHYITPYTFGGGTKVEIK

SEQ ID NO: 12 - protein, 107 aa, Artificial Sequence
  Light chain  variant Vk4 for AB0041
DIQMTQSPSSLSASVGDRVTITCKASQDVRNTVAWYQQKPGKAPKLLIYSSSYRNTGVPDRFSGSGSGTDFTLTISSLQAEDVAVYYCQQHYITPYTFGGGTKVEIK

SEQ ID NO: 13 - protein, 10 aa, Artificial Sequence
  Exemplary heavy chain CDR1 region for anti-MMP9 antibodies
GFSLLSYGVH

SEQ ID NO: 14 - protein, 16 aa, Artificial Sequence
  Exemplary heavy chain CDR2 region for anti-MMP9 antibodies
VIWTGGTTNYNSALMS

SEQ ID NO: 15 - protein, 7 aa, Artificial Sequence
  Exemplary heavy chain CDR3 region for anti-MMP9 antibodies
YYYGMDY

SEQ ID NO: 16 - protein, 11 aa, Artificial Sequence
  Exemplary light chain CDR1 region for anti-MMP9 antibodies
KASQDVRNTVA

SEQ ID NO: 17 - protein, 7 aa, Artificial Sequence
  Exemplary light chain CDR2 region for anti-MMP9 antibodies
SSSYRNT

SEQ ID NO: 18 - protein, 9 aa, Artificial Sequence
  Exemplary light chain CDR3 region for anti-MMP9 antibodies
QQHYITPYT

SEQ ID NO: 19 - DNA, 345 bp, Artificial Sequence
  Polynucleotide encoding heavy chain  variant VH1 for AB0041
caggtgcagctgcaggaatccggccctggcctggtcaagccctccgagacactgtccctgacctgcaccgtgtccggcttctccctgctgtcctacggcgtgcactgggtccgacagcctccagggaagggcctggaatggctgggcgtgatctggaccggcggcaccaccaactacaactccgccctgatgtcccggctgaccatctccaaggacgactccaagtccaccgtgtacctgaagatgaactccctgaaaaccgaggacaccgccatctactactgcgcccggtactactacggcatggactactggggccagggcacctccgtgaccgtgtcctca

SEQ ID NO: 20 - DNA, 345 bp, Artificial Sequence
  Polynucleotide encoding heavy chain  variant VH2 for AB0041
caggtgcagctgcaggaatccggccctggcctggtcaagccctccgagacactgtccctgacctgcaccgtgtccggcttctccctgctgtcctacggcgtgcactgggtccgacagcctccaggcaaaggcctggaatggctgggcgtgatctggaccggcggcaccaccaactacaactccgccctgatgtcccggctgaccatctccaaggacgactccaagaacaccgtgtacctgaagatgaactccctgaaaaccgaggacaccgccatctactactgcgcccggtactactacggcatggactactggggccagggcaccctggtcaccgtgtcctca

SEQ ID NO: 21 - DNA, 345 bp, Artificial Sequence
  Polynucleotide encoding heavy chain  variant VH3 for AB0041
caggtgcagctgcaggaatccggccctggcctggtcaagccctccgagacactgtccctgacctgcaccgtgtccggcttctccctgctgtcctacggcgtgcactgggtccgacagcctccaggcaaaggcctggaatggctgggcgtgatctggaccggcggcaccaccaactacaactccgccctgatgtcccggttcaccatctccaaggacgactccaagaacaccgtgtacctgaagatgaactccctgaaaaccgaggacaccgccatctactactgcgcccggtactactacggcatggactactggggccagggcaccctggtcaccgtgtcctca

SEQ ID NO: 22 - DNA, 345 bp, Artificial Sequence
  Polynucleotide encoding heavy chain  variant VH4 for AB0041
caggtgcagctgcaggaatccggccctggcctggtcaagccctccgagacactgtccctgacctgcaccgtgtccggcttctccctgctgtcctacggcgtgcactgggtccgacagcctccaggcaaaggcctggaatggctgggcgtgatctggaccggcggcaccaccaactacaactccgccctgatgtcccggttcaccatctccaaggacgactccaagaacaccctgtacctgaagatgaactccctgaaaaccgaggacaccgccatctactactgcgcccggtactactacggcatggactactggggccagggcaccctggtcaccgtgtcctca

SEQ ID NO: 23 - DNA, 321 bp, Artificial Sequence
  Polynucleotide encoding light chain variant Vk1 for AB0041
gacatcgtgatgacccagtcccccagcttcctgtccgcctccgtgggcgacagagtgaccatcacatgcaaggcctctcaggacgtgcggaacaccgtggcctggtatcagcagaaaaccggcaaggcccccaagctgctgatctactcctcctcctaccggaacaccggcgtgcccgaccggtttaccggctctggctccggcaccgactttaccctgaccatcagctccctgcaggccgaggacgtggccgtgtacttctgccagcagcactacatcaccccctacaccttcggcggaggcaccaaggtggaaataaaa

SEQ ID NO: 24 - DNA, 321 bp, Artificial Sequence
  Polynucleotide encoding light chain variant Vk2 for AB0041
gacatcgtgatgacccagtccccctccagcctgtccgcctctgtgggcgacagagtgaccatcacatgcaaggcctctcaggacgtgcggaacaccgtggcctggtatcagcagaagcccggcaaggcccccaagctgctgatctactcctcctcctaccggaacaccggcgtgcccgaccggtttaccggctctggctccggcaccgactttaccctgaccatcagctccctgcaggccgaggacgtggccgtgtacttctgccagcagcactacatcaccccctacaccttcggcggaggcaccaaggtggaaataaaa

SEQ ID NO: 25 - DNA, 321 bp, Artificial Sequence
  Polynucleotide encoding light chain variant Vk3 for AB0041
gacatccagatgacccagtccccctccagcctgtccgcctctgtgggcgacagagtgaccatcacatgcaaggcctcccaggacgtgcggaacaccgtggcctggtatcagcagaagcccggcaaggcccccaagctgctgatctactcctcctcctaccggaacaccggcgtgcccgaccggttctctggctctggaagcggcaccgactttaccctgaccatcagctccctgcaggccgaggacgtggccgtgtacttctgccagcagcactacatcaccccctacaccttcggcggaggcaccaaggtggaaataaaa

SEQ ID NO: 26 - DNA, 321 bp, Artificial Sequence
  Polynucleotide encoding light chain variant Vk4 for AB0041
gacatccagatgacccagtccccctccagcctgtccgcctctgtgggcgacagagtgaccatcacatgcaaggcctctcaggacgtgcggaacaccgtggcctggtatcagcagaagcccggcaaggcccccaagctgctgatctactcctcctcctaccggaacaccggcgtgcccgaccggttctctggctctggaagcggcaccgactttaccctgaccatcagctccctgcaggccgaggacgtggccgtgtactactgccagcagcactacatcaccccctacaccttcggcggaggcaccaaggtggaaataaaa

SEQ ID NO: 27 - protein, 707 aa, Homo sapiens
MSLWQPLVLVLLVLGCCFAAPRQRQSTLVLFPGDLRTNLTDRQLAEEYLYRYGYTRVAEMRGESKSLGPALLLLQKQLSLPETGELDSATLKAMRTPRCGVPDLGRFQTFEGDLKWHHHNITYWIQNYSEDLPRAVIDDAFARAFALWSAVTPLTFTRVYSRDADIVIQFGVAEHGDGYPFDGKDGLLAHAFPPGPGIQGDAHFDDDELWSLGKGVVVPTRFGNADGAACHFPFIFEGRSYSACTTDGRSDGLPWCSTTANYDTDDRFGFCPSERLYTRDGNADGKPCQFPFIFQGQSYSACTTDGRSDGYRWCATTANYDRDKLFGFCPTRADSTVMGGNSAGELCVFPFTFLGKEYSTCTSEGRGDGRLWCATTSNFDSDKKWGFCPDQGYSLFLVAAHEFGHALGLDHSSVPEALMYPMYRFTEGPPLHKDDVNGIRHLYGPRPEPEPRPPTTTTPQPTAPPTVCPTGPPTVHPSERPTAGPTGPPSAGPTGPPTAGPSTATTVPLSPVDDACNVNIFDAIAEIGNQLYLFKDGKYWRFSEGRGSRPQGPFLIADKWPALPRKLDSVFEEPLSKKLFFFSGRQVWVYTGASVLGPRRLDKLGLGADVAQVTGALRSGRGKMLLFSGRRLWRFDVKAQMVDPRSASEVDRMFPGVPLDTHDVFQYREKAYFCQDRFYWRVSSRSELNQVDQVGYVTYDILQCPED

SEQ ID NO: 28 - protein, 688 aa, Homo sapiens
APRQRQSTLVLFPGDLRTNLTDRQLAEEYLYRYGYTRVAEMRGESKSLGPALLLLQKQLSLPETGELDSATLKAMRTPRCGVPDLGRFQTFEGDLKWHHHNITYWIQNYSEDLPRAVIDDAFARAFALWSAVTPLTFTRVYSRDADIVIQFGVAEHGDGYPFDGKDGLLAHAFPPGPGIQGDAHFDDDELWSLGKGVVVPTRFGNADGAACHFPFIFEGRSYSACTTDGRSDGLPWCSTTANYDTDDRFGFCPSERLYTRDGNADGKPCQFPFIFQGQSYSACTTDGRSDGYRWCATTANYDRDKLFGFCPTRADSTVMGGNSAGELCVFPFTFLGKEYSTCTSEGRGDGRLWCATTSNFDSDKKWGFCPDQGYSLFLVAAHEFGHALGLDHSSVPEALMYPMYRFTEGPPLHKDDVNGIRHLYGPRPEPEPRPPTTTTPQPTAPPTVCPTGPPTVHPSERPTAGPTGPPSAGPTGPPTAGPSTATTVPLSPVDDACNVNIFDAIAEIGNQLYLFKDGKYWRFSEGRGSRPQGPFLIADKWPALPRKLDSVFEEPLSKKLFFFSGRQVWVYTGASVLGPRRLDKLGLGADVAQVTGALRSGRGKMLLFSGRRLWRFDVKAQMVDPRSASEVDRMFPGVPLDTHDVFQYREKAYFCQDRFYWRVSSRSELNQVDQVGYVTYDILQCPED

SEQ ID NO: 29 - protein, 19 aa, Homo sapiens
MSLWQPLVLVLLVLGCCFA

SEQ ID NO: 30 - protein, 470 aa, Artificial Sequence
  Antibody heavy chain (IgG2b) produced by M4 hybridoma
MAVLVLFLCLVAFPSCVLSQVQLKESGPGLVAPSQSLSITCTVSGFSLLSYGVHWVRQPPGKGLEWLGVIWTGGSTNYNSALMSRLSISKDDSKSQVFLKMNSLQTDDTAMYYCARYYYAMDYWGQGTSVTVSSAKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVRISWFVNNVEVHTAQTQTHREDYNSTIRVVSALPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLDIKTSKWEKTDSFSCNVRHEGLKNYYLKKTISRSPGK

SEQ ID NO: 31 - protein, 234 aa, Artificial Sequence
  Antibody light chain (kappa) produced by M4 hybridoma
MESQIQVFVFVFLWLSGVDGDIVMTQSHKFMFTSVGDRVSITCKASQDVRNTVAWYQQKTGQSPKLLIYSASYRNTGVPDRFTGSISGTDFTFTISSVQAEDLALYYCQQHYSTPYTFGGGTKLEVKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 32 - protein, 115 aa, Artificial Sequence
  Antibody variable heavy chain produced by M4 hybridoma
QVQLKESGPGLVAPSQSLSITCTVSGFSLLSYGVHWVRQPPGKGLEWLGVIWTGGSTNYNSALMSRLSISKDDSKSQVFLKMNSLQTDDTAMYYCARYYYAMDYWGQGTSVTVSS

SEQ ID NO: 33 - protein, 107 aa, Artificial Sequence
  Antibody variable light chain produced by M4 hybridoma
DIVMTQSHKFMFTSVGDRVSITCKASQDVRNTVAWYQQKTGQSPKLLIYSASYRNTGVPDRFTGSISGTDFTFTISSVQAEDLALYYCQQHYSTPYTFGGGTKLEVK

SEQ ID NO: 34 - protein, 10 aa, Artificial Sequence
  Variable heavy chain CDR1 produced by M4 hybridoma
GFSLLSYGVH

SEQ ID NO: 35 - protein, 16 aa, Artificial Sequence
  Variable heavy chain CDR2 produced by M4 hybridoma
VIWTGGSTNYNSALMS

SEQ ID NO: 36 - protein, 7 aa, Artificial Sequence
  Variable heavy chain CDR3 produced by M4 hybridoma
YYYAMDY

SEQ ID NO: 37 - protein, 11 aa, Artificial Sequence
  Variable light chain CDR1 produced by M4 hybridoma
KASQDVRNTVA

SEQ ID NO: 38 - protein, 7 aa, Artificial Sequence
  Variable light chain CDR2 produced by M4 hybridoma
SASYRNT

SEQ ID NO: 39 - protein, 9 aa, Artificial Sequence
  Variable light chain CDR3 produced by M4 hybridoma
QQHYSTPYT

SEQ ID NO: 40 - protein, 229 aa, Artificial Sequence
  Antibody light chain (kappa) produced by M12 hybridoma
QVFVYMLLWLSGVDGDIVMTQSQKFMSTSVGDRVSVTCKASQNVGTNVAWYQQKPGQSPKALIYSASYRFSGVPDRFTGSGSGTDFTLTISNVQSEDLAEYFCQQYNSYPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 41 - protein, 107 aa, Artificial Sequence
  Antibody variable light chain produced by M12 hybridoma
DIVMTQSQKFMSTSVGDRVSVTCKASQNVGTNVAWYQQKPGQSPKALIYSASYRFSGVPDRFTGSGSGTDFTLTISNVQSEDLAEYFCQQYNSYPYTFGGGTKLEIK

SEQ ID NO: 42 - protein, 11 aa, Artificial Sequence
  Variable light chain CDR1 produced by M12 hybridoma
KASQNVGTNVA

SEQ ID NO: 43 - protein, 7 aa, Artificial Sequence
  Variable light chain CDR2 produced by M12 hybridoma
SASYRFS

SEQ ID NO: 44 - protein, 9 aa, Artificial Sequence
  Variable light chain CDR3 produced by M12 hybridoma
QQYNSYPYT

SEQ ID NO: 45 - protein, 233 aa, Artificial Sequence
  Mouse antibody light chain (kappa) AB0046
MSSAQFLGLLLLCFQGTRCDIQMTQTTSSLSASLGDRVTISCSASQGISNYLNWYQQKPDGTFKLLIYYTSILHSGVPSRFSGSGSGTDYSLTISNLEPEDIATYYCQQYGWLPRTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 46 - protein, 460 aa, Artificial Sequence
  Mouse antibody IgG1 heavy chain AB0046
MGWSSIILFLVATATGVHSQVQLQQPGSVLVRPGASVKLSCTASGYTFTSYWMNWVKQRPGQGLEWIGEIYPISGRTNYNEKFKVKATLTVDTSSSTAYMDLNSLTSEDSAVYYCARSRANWDDYWGQGTTLTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 47 - protein, 117 aa, Artificial Sequence
  Mouse antibody variable region of IgG1 heavy chain AB0046
QVQLQQPGSVLVRPGASVKLSCTASGYTFTSYWMNWVKQRPGQGLEWIGEIYPISGRTNYNEKFKVKATLTVDTSSSTAYMDLNSLTSEDSAVYYCARSRANWDDYWGQGTTLTVSS

SEQ ID NO: 48 - protein, 107 aa, Artificial Sequence
  Mouse antibody variable region of kappa light chain AB0046
DIQMTQTTSSLSASLGDRVTISCSASQGISNYLNWYQQKPDGTFKLLIYYTSILHSGVPSRFSGSGSGTDYSLTISNLEPEDIATYYCQQYGWLPRTFGGGTKLEIK

SEQ ID NO: 49 - protein, 461 aa, Artificial Sequence
  Heavy chain of AB0045 antibody
MGWSLILLFLVAVATRVHSQVQLQESGPGLVKPSETLSLTCTVSGFSLLSYGVHWVRQPPGKGLEWLGVIWTGGTTNYNSALMSRFTISKDDSKNTVYLKMNSLKTEDTAIYYCARYYYGMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 50 - protein, 234 aa, Artificial Sequence
  Light chain of AB0045 antibody
MRVPAQLLGLLLLWLPGARCDIQMTQSPSSLSASVGDRVTITCKASQDVRNTVAWYQQKPGKAPKLLIYSSSYRNTGVPDRFSGSGSGTDFTLTISSLQAEDVAVYYCQQHYITPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 51 - protein, 148 aa, Artificial Sequence
  Antibody light chain (kappa) produced by M4 hybridoma
MESQIQVFVFVFLWLSGVDGDIVMTQSHKFMFTSVGDRVSITCKASQDVRNTVAWYQQKTGQSPKLLIYSASYRNTGVPDRFTGSISGTDFTFTISSVQAEDLALYYCQQHYSTPYTFGGGTKLEVKRADAAPTVSIFPPSSEQLTSG

SEQ ID NO: 52 - protein, 148 aa, Artificial Sequence
  Mouse monoclonal anti-MMP9 antibody - AB0041 light chain
MESQIQVFVFVFLWLSGVDGDIVMTQSHKFMSTSVGDRVSITCKASQDVRNTVAWYQQKTGQSPKLLIYSSSYRNTGVPDRFTGSGSGTDFTFTISSVQAEDLAVYFCQQHYITPYTFGGGTKLEIKRADAAPTVSIFPPSSEQLTSG

SEQ ID NO: 53 - protein, 143 aa, Artificial Sequence
  Antibody light chain (kappa) produced by M12 hybridoma
QVFVYMLLWLSGVDGDIVMTQSQKFMSTSVGDRVSVTCKASQNVGTNVAWYQQKPGQSPKALIYSASYRFSGVPDRFTGSGSGTDFTLTISNVQSEDLAEYFCQQYNSYPYTFGGGTKLEIKRADAAPTVSIFPPSSEQLTSG

SEQ ID NO: 54 - protein, 180 aa, Artificial Sequence
  Antibody heavy chain (IgG2b) produced by M4 hybridoma
MAVLVLFLCLVAFPSCVLSQVQLKESGPGLVAPSQSLSITCTVSGFSLLSYGVHWVRQPPGKGLEWLGVIWTGGSTNYNSALMSRLSISKDDSKSQVFLKMNSLQTDDTAMYYCARYYYAMDYWGQGTSVTVSSAKTTPPSVYPLAPGCGDTTGSSVTLGCLVKGYFPESVTVTWNSGSL

SEQ ID NO: 55 - protein, 180 aa, Artificial Sequence
  Mouse monoclonal anti-MMP9 antibody - AB0041 heavy chain
MAVLVLFLCLVAFPSCVLSQVQLKESGPGLVAPSQSLSITCTVSGFSLLSYGVHWVRQPPGKGLEWLGVIWTGGTTNYNSALMSRLSISKDDSKSQVFLKMNSLQTDDTAIYYCARYYYGMDYWGQGTSVTVSSAKTTPPSVYPLAPGCGDTTGSSVTLGCLVKGYFPESVTVTWNSGSL

SEQ ID NO: 56 - protein, 442 aa, Artificial Sequence
  Heavy chain of AB0045 antibody
QVQLQESGPGLVKPSETLSLTCTVSGFSLLSYGVHWVRQPPGKGLEWLGVIWTGGTTNYNSALMSRFTISKDDSKNTVYLKMNSLKTEDTAIYYCARYYYGMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 57 - protein, 214 aa, Artificial Sequence
  Light chain of AB0045 antibody
DIQMTQSPSSLSASVGDRVTITCKASQDVRNTVAWYQQKPGKAPKLLIYSSSYRNTGVPDRFSGSGSGTDFTLTISSLQAEDVAVYYCQQHYITPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 58 - protein, 451 aa, Artificial Sequence
  Mouse monoclonal anti-MMP9 antibody - AB0041 heavy chain
QVQLKESGPGLVAPSQSLSITCTVSGFSLLSYGVHWVRQPPGKGLEWLGVIWTGGTTNYNSALMSRLSISKDDSKSQVFLKMNSLQTDDTAIYYCARYYYGMDYWGQGTSVTVSSAKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVRISWFVNNVEVHTAQTQTHREDYNSTIRVVSALPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLDIKTSKWEKTDSFSCNVRHEGLKNYYLKKTISRSPGK

SEQ ID NO: 59 - protein, 214 aa, Artificial Sequence
  Mouse monoclonal anti-MMP9 antibody - AB0041 light chain
DIVMTQSHKFMSTSVGDRVSITCKASQDVRNTVAWYQQKTGQSPKLLIYSSSYRNTGVPDRFTGSGSGTDFTFTISSVQAEDLAVYFCQQHYITPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

(19) United States
(12) Patent Application Publication
(10) Pub. No.: US 2015/0239988 A1
(43) Pub. Date: Aug. 27, 2015

(54) ANTIBODIES TO MATRIX METALLOPROTEINASE 9 AND METHODS OF USE THEREOF

(71) Applicant: Gilead Biologics, Inc., Foster City, CA (US)

(72) Inventors: David L. Gossage, Seattle, WA (US); Joseph H. Lin, San Mateo, CA (US); Victoria Smith, Burlingame, CA (US); Maria Vaysberg, Los Altos, CA (US)

(21) Appl. No.: 14/631,574

(22) Filed: Feb. 25, 2015

Related U.S. Application Data
(60) Provisional application No. 61/945,737, filed on Feb. 27, 2014.

(51) Int. Cl. C07K 16/40; A61K 39/395; A61K 45/06

(52) U.S. Cl. CPC C07K 16/40; A61K 45/06; A61K 39/3955; C07K 2317/565; C07K 2317/56; C07K 2317/34; C07K 2317/24; A61K 2039/505

(57) ABSTRACT

The present disclosure provides compositions and methods of use involving binding proteins, e.g., antibodies and antigen binding fragments thereof, that bind to the matrix metalloproteinase-9 (MMP9) protein (MMP9 is also known as gelatinase-B), such as where the binding proteins comprise an immunoglobulin (Ig) heavy chain (or functional fragment thereof) and an Ig light chain (or functional fragment thereof).

### CROSS-REFERENCE TO RELATED APPLICATIONS

This application claims benefit under 35 U.S.C. §119(e) to U.S. Provisional Application Ser. No. 61/945,737, filed on Feb. 27, 2014, the entirety of which is incorporated herein by reference.

### REFERENCE TO SEQUENCE LISTING SUBMITTED VIA EFS-WEB

The entire content of the following electronic submission of the sequence listing via the USPTO EFS-WEB server, as authorized and set forth in MPEP §1730 II.B.2(a) (C), is incorporated herein by reference in its entirety for all purposes. The sequence listing is identified on the electronically filed text for as follows:

File Name: 1076PC_Sequence_Listing; Date of Creation: Feb. 23, 2015; Size (bytes): 86,016 bytes

### FIELD

This disclosure is in the field of extracellular enzymes, extracellular matrix enzymes, proteases and immunology.

### BACKGROUND

Matrix metalloproteinases (MMPs) belong to a family of extracellular enzymes involved in forming and remodeling the extracellular matrix. These enzymes contain a conserved catalytic domain in which a zinc atom is coordinated by three histidine residues. Over 20 members of this family are known, organized into a number of groups including collagenases, gelatinases, stromelysins, matrilysins, enamelysins and membrane MMPs.

MMP2 and MMP9 belong to the gelatinase group of matrix metalloproteinases. Besides containing signal peptide, propeptide, catalytic, zinc-binding and heamopexin-like domains common to most MMPs, the gelatinases also contain a plurality of fibronectin-like domains and an O-glycosylated domain. MMPs are involved in a number of diseases. Inhibitors of MMPs have not been entirely satisfactory, in part related to specificity and efficacy. Thus, there is a need for specific and effective MMP inhibitors.

Despite active drug development for decades and the FDA approval of a new therapeutic class for COPD (phosphodiesterase-4 inhibitor roflumilast) in 2011, there is persistent unmet need in COPD as measured by symptoms, exacerbations, hospitalizations, and mortality. No pharmacologic therapy has been shown to alter disease progression or mortality. There are numerous active development programs with nonbronchodilator mechanisms for COPD; however, few have the potential to alter both inflammation and disease progression with acceptable safety. Thus, there is a need for treatments effective in such diseases, particularly for subjects in which available therapeutics have been ineffective.

### SUMMARY

Provided herein is a method for treating or preventing chronic obstructive pulmonary disease (COPD) in a subject, comprising administering to the subject an effective amount of an Matrix Metalloproteinase 9 (MMP9) binding protein comprising an immunoglobulin heavy chain polypeptide, or functional fragment thereof, and an immunoglobulin light chain polypeptide, or functional fragment thereof, wherein the MMP9 binding protein specifically binds MMP9. In some embodiments, the MMP9 binding protein is an antibody or antigen binding fragment thereof that binds to matrix metalloproteinase-9 (MMP9) protein (also known as gelatinase-B). The antibody that binds to MMP9 or fragment (e.g., antigen binding fragment) thereof typically contains an immunoglobulin (Ig) heavy chain (or functional fragment thereof) and an Ig light chain (or functional fragment thereof). The heavy chain is typically an IgG, such as an IgG1 or IgG4, or modified version thereof. The light chain typically is a kappa chain.

In some embodiments, among the MMP9 binding proteins, e.g., antibodies, are those that bind specifically to MMP9 and not to other, related matrix metalloproteinases. Such MMP9 binding proteins find use in applications in which it is necessary or desirable to obtain specific modulation (e.g., inhibition) of MMP9, e.g., without directly affecting the activity of other matrix metalloproteinases. Thus, in certain embodiments of the present disclosure an anti-MMP9 antibody or antigen binding fragment thereof is a specific inhibitor of the activity of MMP9. In some aspects, the MMP9 binding proteins disclosed herein will be useful for inhibition of MMP9 while allowing normal function of other, related matrix metalloproteinases.

In certain embodiments, the antibody that binds to MMP9 or antigen binding fragment thereof that specifically binds to an epitope of MMP9, wherein the epitope comprises an amino acid residue within a region of MMP9, the region comprises residues 104-119, residues 159-166, or residues 191-202 of SEQ ID NO: 27. In certain embodiments, the region consists of residues 104-119, residues 159-166, or residues 191-202 of SEQ ID NO: 27. In one aspect, the epitope comprises E111, D113, R162, or I198 of SEQ ID NO: 27. For example, the epitope may comprise residue R162 of SEQ ID NO:27. In certain embodiments, the antibody is a humanized, chimeric, or human antibody.

The antibodies and fragments can be described with reference to their amino acid sequences or portions thereof, and/or various functions such as binding specificity to MMP9 or particular epitopes thereof or the ability to compete for binding with particular antibodies, and/or activity, such as the ability to inhibit MMP9, e.g., non-competitively.

In certain embodiments, the antibody that binds to MMP9 or the antigen binding fragment thereof comprises a VH region comprising a CDR with an amino acid sequence selected from the group consisting of SEQ ID NOs: 13, 14, and 15; and a VL region having a CDR with an amino acid sequence selected from the group consisting of SEQ ID NOs: 16, 17, and 18. In certain embodiments, the antibody that binds to MMP9 or the antigen binding fragment thereof comprises a VH region comprising three CDRs comprising the amino acid sequences of SEQ ID NOs: 13, 14, and 15; and a VL region comprising three CDRs comprising the amino acid sequences of SEQ ID NOs: 16, 17, and 18. In certain embodiments, the VH region has the amino acid sequence selected from the group consisting of SEQ ID NOs: 3, 5, 6, 7, and 8; and the VL region has the amino acid sequence selected from the group consisting of SEQ ID NOs: 4, 9, 10, 11, and 12.

In certain embodiments, the antibody that binds to MMP9 or the antigen binding fragment thereof comprises a VH region comprising the amino acid sequence set forth in SEQ ID NO: 7 and a VL region comprising the amino acid sequence set forth in SEQ ID NO: 12.

In certain embodiments, the antibody that binds to MMP9 comprises a heavy chain comprising the amino acid sequence set forth in SEQ ID NO: 49 without the signal peptide and a light chain comprising the amino acid sequence set forth in SEQ ID NO: 50 without the signal sequence. In some embodiments, the antibody that binds to MMP9 comprises a heavy chain comprising the amino acid sequence set forth in SEQ ID NO: 56 and a light chain comprising the amino acid sequence set forth in SEQ ID NO: 57.

In certain embodiments, the antibody that binds to MMP9 or the antigen binding fragment thereof comprises a VH region comprising the amino acid sequence set forth in SEQ ID NO: 47 and a VL region comprising the amino acid sequence set forth in SEQ ID NO: 48.

In certain embodiments, the antibody that binds to MMP9 comprises a light chain comprising the amino acid sequence set forth in SEQ ID NO: 45 without the signal peptide and a heavy chain comprising the amino acid sequence set forth in SEQ ID NO: 46 without the signal peptide. In some embodiments, the antibody that binds to MMP9 comprises a heavy chain comprising the amino acid sequence set forth in SEQ ID NO: 58 and a light chain comprising the amino acid sequence set forth in SEQ ID NO: 59.

In certain embodiments, the antibody that binds to MMP9 or the antigen binding fragment thereof is any antibody or antigen binding fragment thereof described herein. In one embodiment, the method for treating or preventing COPD in a subject comprising administering to the subject an effective amount of an anti-MMP9 antibody or antigen binding fragment thereof. In some embodiments, the anti-MMP9 antibody or antigen binding fragment thereof may bind to an epitope of MMP9, wherein the epitope comprises amino acid residues 104-119, residues 159-166, or residues 191-202 of SEQ ID NO: 27; or amino acid residues E111, D113, R162, or I198 of SEQ ID NO: 27. In certain embodiments, the anti-MMP9 antibody or antigen binding fragment thereof may compete for binding to MMP9 with a protein or an antibody, wherein the protein or the antibody may bind to amino acid residues 104-119, residues 159-166, or residues 191-202 of SEQ ID NO: 27; or amino acid residues E111, D113, R162, or I198 of SEQ ID NO: 27. In certain other embodiments, the anti-MMP9 antibody or antigen binding fragment thereof may compete for binding to MMP9 with a protein or an antibody, wherein the protein or the antibody having about 95%, 96%, 97%, 98%, 99% or greater identify to the amino acid sequence selected from the group consisting of SEQ ID NOs: 7, 12, 13, 14, 15, 16, 17, and 18. In further embodiments, the anti-MMP9 antibody or antigen binding fragment thereof may comprise a heavy chain variable (VH) region comprising a complementarity-determining region (CDR) having an amino acid sequence selected from the group consisting of SEQ ID NOs: 13, 14, and 15; a light chain variable (VL) region having a CDR having an amino acid sequence selected from the group consisting of SEQ ID NOs: 16, 17, and 18; a VH region comprising three CDRs (i.e. CDR 1-3) comprising the amino acid sequences set forth in SEQ ID NOs: 13, 14, and 15; or a VL region comprising three CDRs (i.e. CDR 1-3) comprising the amino acid sequences set forth in SEQ ID NOs: 16, 17, and 18. In some further embodiments, the anti-MMP9 antibody or antigen binding fragment thereof may comprise the VH region comprising the amino acid sequence selected from the group consisting of SEQ ID NOs: 3, 5, 6, 7, and 8; the VL region comprising the amino acid sequence selected from the group consisting of SEQ ID NOs: 4, 9, 10, 11, and 12; a heavy chain comprising the amino acid sequence set forth in SEQ ID NOs: 56 or 58; or a light chain comprising the amino acid sequence set forth in SEQ ID NOs: 57 or 59. In certain further embodiments, the anti-MMP9 antibody or antigen binding fragment thereof comprises a VH region comprising the amino acid sequence set forth in SEQ ID NO: 7 and a VL region comprising the amino acid sequence set forth in SEQ ID NO: 12.

In some embodiments, the MMP9 binding protein, e.g., an antibody that binds MMP9 or antigen binding fragment thereof, is in a pharmaceutical composition.

In certain embodiments, the MMP9 binding protein inhibits the enzymatic activity of MMP9. In certain embodiments, the inhibition of enzymatic activity is non-competitive.

In certain embodiments, the antibody that binds to MMP9 or the antigen binding fragment thereof, or the pharmaceutical composition thereof, is administered to the subject at a dose from about 25 mg to about 800 g. In some examples, the antibody that binds to MMP9 or the antigen binding fragment thereof, or the pharmaceutical composition thereof, is administered to the subject at a dosage of about 200 mg, about 300 mg, or about 400 mg at an interval of one, two, or three weeks, or once every one, two, or three weeks. In certain embodiments, the antibody that binds to MMP9 or the antigen binding fragment thereof is administered to the subject at a dosage of about 400 mg every two weeks. In certain embodiments, the antibody that binds to MMP9 or the antigen binding fragment thereof is administered to the subject at a dosage of about 200 mg every two weeks. In some embodiments, the antibody that binds to MMP9 or the antigen binding fragment thereof is administered to the subject in a two-step procedure: first, a loading dose phase (more frequent dosing to cover the “target sink” or high baseline concentration of MMP9 associated with the disease, wherein the dosing range is administered to the subject at a dosage of about 200 mg, about 300 mg, or about 400 mg every week for an interval of one, two or three weeks, or more frequent dosing to cover the “target sink” or high baseline concentration of MMP9 associated with the disease) and second, once a predictable pK has been established after the loading dose phase, a lower weekly dose such as 150, 125, 100 or 50 mg/week. In some embodiments, the lower weekly dose could be lower on a weekly basis, e.g., 150, 125, 100 or 50 mg/week. In some embodiments, the anti-MMP9 antibody or antigen binding fragment thereof is administered at a dose of about 100 mg, 150 mg, 200 mg, 300 mg, or 400 mg.

In certain embodiments, the antibody that binds to MMP9 or the antigen binding fragment thereof, or the pharmaceutical composition thereof, is administered the interval of one, two or three weeks, or once every one, two, or three weeks. In certain embodiments, the antibody that binds to MMP9 or the antigen binding fragment thereof, or the pharmaceutical composition thereof, is administered intravenously, intradermally, or subcutaneously. In one embodiment, the anti-MMP9 antibody or antigen binding fragment thereof, or the composition or the pharmaceutical formulation thereof is administered subcutaneously. In other embodiment, the anti-MMP9 antibody or antigen binding fragment thereof, or the composition or the pharmaceutical formulation thereof is administered intravenously.

In certain embodiments, the antibody that binds to MMP9 or the antigen binding fragment thereof, or the pharmaceutical composition thereof, is administered alone, as a monotherapy. In certain embodiments, the antibody that binds to MMP9 or the antigen binding fragment thereof, or the pharmaceutical composition thereof, is administered as part of a combination therapy with one or more other therapeutic agents for the treatment of COPD. The therapeutic agents for the treatment of COPD include, but are not limited to 1) Short-acting β2 agonists (such as, for example, salbutamol (albuterol), levalbuterol, fenoterol, terbutaline), 2) short-acting anticholinergics (such as, for example, ipratropium bromide, oxitropium bromide), 3) Long-acting β-2 agonists (such as, for example, formoterol, arfomoterol, indacaterol, salmeterol, tulobuterol), 4) Long-acting anticholinergics (such as, for example, aclidinium bromide, glycopyrronium bromide, tiotropium), 5) Combination short-acting β-2 agonist plus anticholinergics (such as, for example, fenoterol/ipratropium, salbutamol/ipratropium, 6) Inhaled corticosteroids (such as, for example, beclomethasone, budesonide, fluticasone, 7) Combination long-acting β-2 agonists plus corticosteroids (such as, for example, formoterol/budesonide, formoterol/mometasone, salmeterol/fluticasone), 8) Methylxanthines (such as, for example, aminophylline, theophylline), 9) Phosphosdiesterase-4 inhibitors (such as, for example, roflumilast), 10) Systemic corticosteroids (such as, for example, prednisone, methylprednisolone). The one or more other therapeutic agents can be administered concurrently or sequentially with the antibody that binds MMP9 or an antigen binding fragment thereof. In one embodiment, the MMP9 antibody or antigen binding fragment thereof may be combined with the therapeutic agents selected from the group consisting of short-acting β-2 agonists, short-acting anticholinergics, long-acting β-2 agonists, long-acting anticholinergics, and a combination thereof. Any of the combinations of anti-MMP9 antibody or antigen binding fragment thereof and the one or more therapeutic agents may be further combined with β-2 agonist, anticholinergics, inhaled corticosteroids, systemic corticosteroids, methylxanthines, phosphosdiesterase-4 inhibitors, or a combination thereof.

In certain embodiments, the subject has chronic bronchitis, emphysema, or both. In certain embodiments, the subject has been diagnosed with COPD. In certain embodiments, the subject has diagnosis of COPD per Global Initiative for Chronic Obstructive Lung Disease (GOLD) guidelines for at least 6 months. In some embodiments, the subject has post-bronchodilator forced expiratory volume in one second (FEV₁)≧40% predicted. In some embodiments, the subject has post-bronchodilator forced expiratory volume in one second (FEV₁)≧40% and ≦80% predicted. In some embodiments, the subject has no changes in COPD medications within 30 days. In other embodiments, the subject is diagnosed with chronic bronchitis, emphysema, or COPD having, one, two or more exacerbations in a year. In one embodiment, the subject is diagnosed with COPD having two or more exacerbations in a year and reduced lung function prior to the treatments of the present application.

In certain embodiments, the subject does not have clinically significant active infection. In certain embodiments, the subject does not have a positive QuantiFERON-TB GOLD test. In certain embodiments, the subject does not have any serious cardiac event such as myocardial infarction, unstable or life-threatening arrhythmia, hospitalization for cardiac failure within 6 months or any significant or new electrocardiogram (ECG) finding. In certain embodiments, the subject has not been hospitalized for a respiratory event such as, but not limited to, COPD, pneumonia, bronchiolitis, within the previous 6 months. In certain embodiments, the subject does not have chronic lung disease other than COPD (such as: asthma, cystic fibrosis or fibrotic disease, α-1-antitrypsin deficiency, interstitial lung disease, pulmonary thromboembolic disease, or bronchiectasis). In certain embodiments, the subject does not exhibit chronic use of systemic corticosteroids and/or treatment with systemic corticosteroids for an acute exacerbation of COPD (AECOPD) event, or other medical condition not requiring hospitalization, within 90 days. In certain embodiments, the subject has not been treated with antibiotics for an AECOPD event, or other medical condition not requiring hospitalization within 90 days of randomization, or any minor medical event not requiring hospitalization within 14. In certain embodiments, the subject has not been treated with any marketed or investigational biologic within 5 half-lives of the molecule or if unknown within 90 days. In certain embodiments the subject is not currently on nonbiologic immune modulator medications (such as: azathioprine, cyclosporine, hydroxychloroquine, leflunomide, methotrexate, mycophenolate mofetil, sulfasalazine, tofacitinib) within 90 days.

It is to be understood that one, some, or all of the properties of the various embodiments described herein may be combined to form other embodiments of the present invention. These and other aspects of the invention will become apparent to one of skill in the art. These and other embodiments of the invention are further described by the detailed description that follows.

### BRIEF DESCRIPTION OF THE DRAWINGS

FIG. 1 shows the amino acid sequence of the heavy chain variable region of a mouse monoclonal anti-MMP9 antibody (AB0041), along with the amino acid sequences of humanized variants of heavy chain (VH1-VH4), aligned to show differences in framework amino acid sequence resulting from humanization. CDRs are shown in italics, and amino acids that are different in the humanized variants, compared to the parent mouse monoclonal, are underlined.

FIG. 2 shows the amino acid sequence of the light chain variable region of a mouse monoclonal anti-MMP9 antibody (AB0041), along with the amino acid sequences of humanized variants of this light chain (VH1-VH4), aligned to show differences in framework amino acid sequence resulting from humanization. CDRs are shown in italics, and amino acids that are different in the humanized variants, compared to the parent mouse monoclonal, are underlined.

FIG. 3 shows a schematic diagram of the MMP9 protein.

FIG. 4 shows a comparison between the amino acid sequences of the heavy and light chains of antibodies designated AB0041, M4, and M12. SEQ ID NOs: 51, 52, and 53 illustrate the amino acid sequences of the light chains of M4, AB0041, and M12, respectively; and SEQ ID NOs: 54 and 55 illustrate the amino acid sequences of the heavy chains of M4 and AB0041, respectively.

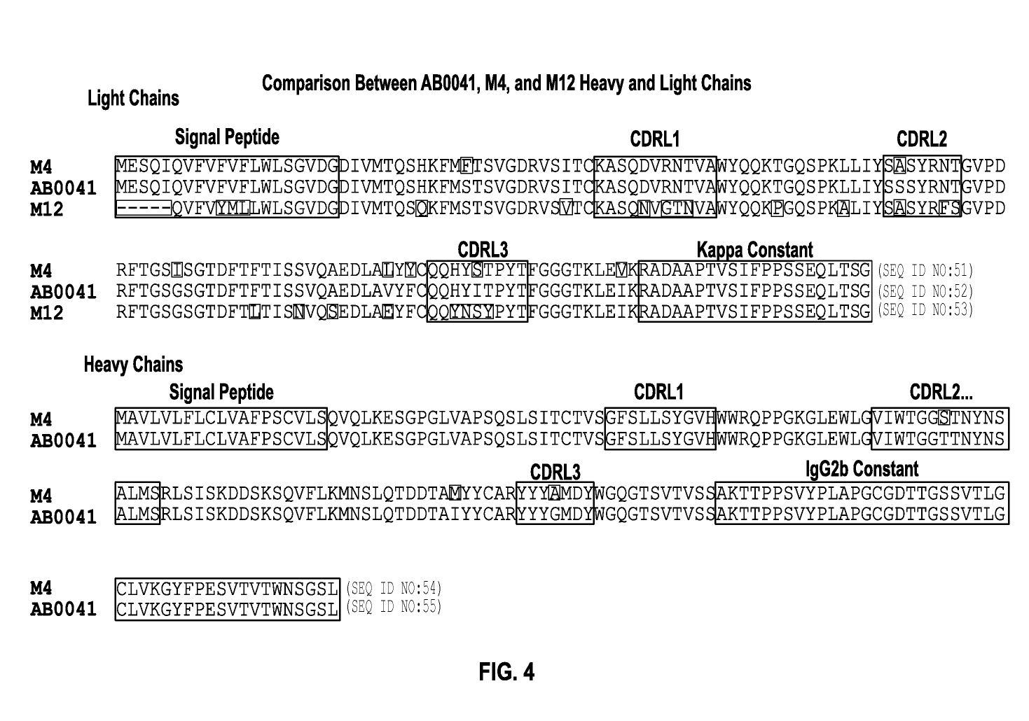

### DETAILED DESCRIPTION

Practice of the present disclosure employs, unless otherwise indicated, standard methods and conventional techniques in the fields of cell biology, toxicology, molecular biology, biochemistry, cell culture, immunology, oncology, recombinant DNA and related fields as are within the skill of the art. Such techniques are described in the literature and thereby available to those of skill in the art. See, for example, Alberts, B. et al., “Molecular Biology of the Cell,” 5^{th} edition, Garland Science, New York, N.Y., 2008; Voet, D. et al. “Fundamentals of Biochemistry: Life at the Molecular Level,” 3^{rd} edition, John Wiley & Sons, Hoboken, N.J., 2008; Sambrook, J. et al., “Molecular Cloning: A Laboratory Manual,” 3^{rd} edition, Cold Spring Harbor Laboratory Press, 2001; Ausubel, F. et al., “Current Protocols in Molecular Biology,” John Wiley & Sons, New York, 1987 and periodic updates; Freshney, R. I., “Culture of Animal Cells: A Manual of Basic Technique,” 4^{th} edition, John Wiley & Sons, Somerset, N.J., 2000; and the series “Methods in Enzymology,” Academic Press, San Diego, Calif. See also, for example, “Current Protocols in Immunology,” (R. Coico, series editor), Wiley, last updated August 2010.

Abnormal activity of certain MMPs plays a role in tumor growth, metastasis, inflammation, autoimmunity, and vascular disease. See, for example, Hu et al. (2007) Nature Reviews: Drug Discovery 6:480-498. It can be desirable to inhibit the activity of one or more MMPs in certain therapeutic settings. However, the activity of certain other MMPs, e.g., MMP2, is often required for normal function and/or is protective against disease. Since most MMP inhibitors are targeted to the conserved catalytic domain and, as a result, inhibit a number of different MMPS, use of available MMP inhibitors has caused side effects due to the inhibition of essential, non-pathogenically-related MMPs. These side effects may likely be also due to general zinc chelation caused by many of these inhibitors, including inhibiting zinc-requiring enzymes more broadly.

Challenges associated with developing inhibitors specific to a particular MMP or select MMPs relate to the fact that inhibition of enzymatic activity via substrate-competitive mechanisms generally requires that the inhibitor be targeted to the catalytic domain. Homologies in MMP catalytic domains can cause inhibitors to react with more than one MMP. Among the provided embodiments are agents, including therapeutic reagents, such as antibodies and antigen binding fragments thereof, that specifically inhibit the catalytic activity of a single MMP or a select plurality of MMPs, such as MMP9 and that do not react with or inhibit certain other MMPs or any other MMPs. Also among the provided embodiments are methods and uses of the same for treatment of chronic obstructive pulmonary disorder (COPD). In certain embodiments, the MMP9 binding protein(s) of this disclosure bind(s) the general large catalytic domain, but is not binding in the substrate pocket and appears to be acting via other, allosteric mechanisms (e.g. the MMP9 binding protein of this disclosure does not compete with substrate for binding, and inhibits independently of the presence of substrate or substrate concentration).

Provided herein are methods for treating or preventing chronic obstructive pulmonary disease (COPD) in a subject, comprising administering to the subject an effective amount of an Matrix Metalloproteinase 9 (MMP9) binding protein comprising an immunoglobulin heavy chain polypeptide, or functional fragment thereof, and an immunoglobulin light chain polypeptide, or functional fragment thereof, wherein the MMP9 binding protein specifically binds MMP9. Also provided are compositions for use in the treatment or prevention of COPD, wherein the compositions comprise Matrix Metalloproteinase 9 (MMP9) binding protein comprising an immunoglobulin heavy chain polypeptide, or functional fragment thereof, and an immunoglobulin light chain polypeptide, or functional fragment thereof, wherein the MMP9 binding protein specifically binds MMP9. Also provided is use of a Matrix Metalloproteinase 9 (MMP9) binding protein in the manufacture of a medicament for the treatment or prevention of COPD, wherein the MMP9 protein comprises an immunoglobulin heavy chain polypeptide, or functional fragment thereof, and an immunoglobulin light chain polypeptide, or functional fragment thereof, and wherein the MMP9 binding protein specifically binds MMP9. Any MMP9 binding proteins described herein (e.g., an antibody that binds MMP9 or an antigen binding fragment thereof) can be used in the methods described herein.

As used herein, the singular form “a”, “an”, and “the” includes plural references unless indicated otherwise.

Reference to “about” a value or parameter herein refers to the usual error range for the respective value readily known to the skilled person in this technical field. Reference to “about” a value or parameter herein includes (and describes) aspects that are directed to that value or parameter per se. For example, description referring to “about X” includes description of “X.” In certain embodiments, the term “about” includes the indicated amount±1% to 10%. In other embodiments, the term “about” includes the indicated amount±5%. In certain other embodiments, the term “about” includes the indicated amount±1%. In certain other embodiments, the term “about” includes the indicated amount±10%.

It is understood that aspects and embodiments of the invention described herein include “comprising,” “consisting,” and “consisting essentially of” aspects and embodiments.

All references cited herein, including patent applications and publications, are hereby incorporated by reference in their entirety.

### MMP9 Binding Proteins

MMP9 degrades basement membrane collagen and other extracellular matrix (ECM) components. Kessenbrock K, et al., “Matrix metalloproteinases: regulators of the tumor microenvironment.” Cell 2010; 141 (1):52-67. Matrix degradation contributes to pathology in multiple diseases, including arthritis, cancer, and ulcerative colitis. Roy R, et al., “Matrix metalloproteinases as novel biomarkers and potential therapeutic targets in human cancer.” J Clin Oncol 2009; 27 (31):5287-97. Broad-spectrum matrix metalloproteinase inhibitors such as Marimastat are efficacious in animal models of inflammation and cancer (Watson S A, et al., “Inhibition of tumour growth by marimastat in a human xenograft model of gastric cancer: relationship with levels of circulating CEA.” Br J Cancer 1999; 81 (1):19-23; Sykes A P, et al., “The effect of an inhibitor of matrix metalloproteinases on colonic inflammation in a trinitrobenzenesulphonic acid rat model of inflammatory bowel disease.” Aliment Pharmacol Ther 1999; 13 (11):1535-42.). Such pan inhibitors, however, can cause musculoskeletal side effects including joint stiffness, inflammation, and pain in the hands, arms, and shoulders, collectively referred to as musculoskeletal syndrome (MSS), typically at or near efficacious dose levels of Marimastat in humans. Peterson J T. “The importance of estimating the therapeutic index in the development of matrix metalloproteinase inhibitors.” Cardiovasc Res 2006; 69 (3):677-87; Tierney G M, et al. “A pilot study of the safety and effects of the matrix metalloproteinase inhibitor marimastat in gastric cancer.” Eur J Cancer 1999; 35 (4):563-8; Wojtowicz-Praga S, et al. “Phase I trial of Marimastat, a novel matrix metalloproteinase inhibitor, administered orally to patients with advanced lung cancer.” J Clin Oncol 1998; 16 (6):2150-6. The symptoms are dose- and time-dependent, and reversible shortly after cessation of treatment with the pan-MMP inhibitor. Wojtowicz-Praga S, 1998; Nemunaitis J, et al., “Combined analysis of studies of the effects of the matrix metalloproteinase inhibitor marimastat on serum tumor markers in advanced cancer: selection of a biologically active and tolerable dose for longer-term studies.” Clin Cancer Res 1998; 4 (5):1101-9; Hutchinson J W et al., “Dupuytren's disease and frozen shoulder induced by treatment with a matrix metalloproteinase inhibitor.” The Journal of bone and joint surgery. British volume 1998; 80 (5):907-8. Marimastat and other pan-MMP inhibitors of the same class are zinc chelators. Peterson J T, 2006. The homozygous MMP9 knockout mouse displays no MSS-like symptoms or MSS-like tissue changes. Vu T H, et al., “MMP-9/gelatinase B is a key regulator of growth plate angiogenesis and apoptosis of hypertrophic chondrocytes.” Cell 1998; 93 (3):411-22.

The present disclosure provides binding proteins, e.g., antibodies and fragments (e.g., antigen binding fragments) thereof, that bind to the matrix metalloproteinase-9 (MMP9) protein (MMP9 is also known as gelatinase-B), e.g., human MMP9, such as the human MMP9 having an amino acid sequence set forth in SEQ ID NO: 27 or SEQ ID NO: 28. The binding proteins of the present disclosure generally comprise an immunoglobulin (Ig) heavy chain (or functional fragment thereof) and an Ig light chain (or functional fragment thereof).

The disclosure further provides MMP9 binding proteins that bind specifically to MMP9 and not to other matrix metalloproteinases such as MMP1, MMP2, MMP3, MMP7, MMP9, MMP10, MMP12, and MMP13. Such specific MMP9 binding proteins are thus generally not significantly or detectably crossreactive with non-MMP9 matrix metalloproteinases. MMP9 binding proteins that specifically bind MMP9 find use in applications in which it is necessary or desirable to obtain specific modulation (e.g., inhibition) of MMP9, e.g., without directly affecting the activity of other matrix metalloproteinases.

In certain embodiments of the present disclosure an anti-MMP9 antibody is an inhibitor of the activity of MMP9, and can be a specific inhibitor of MMP9. In particular, the MMP9 binding proteins disclosed herein will be useful for inhibition of MMP9 while allowing normal function of other, related matrix metalloproteinases. “An inhibitor of MMP” or “inhibitor of MMP9 activity” can be an antibody or an antigen binding fragment thereof that directly or indirectly inhibits activity of MMP9, including but not limited to enzymatic processing, inhibiting action of MMP9 on it substrate (e.g., by inhibiting substrate binding, substrate cleavage, and the like), and the like.

In some embodiments, as demonstrated in examples herein, whereas treatment with pan-MMP inhibitors, such as small-molecule pan inhibitors such as Marimastat, result in symptoms of musculoskeletal disease, such as musculoskeletal syndrome (MSS), including substantial effects on gait, posture and willingness to move, and profound histological damage to joints, specific inhibition of MMP9 such as the antibodies or antigen binding fragments thereof in the present application, does not cause such symptoms and does not induce MSS.

The present disclosure also provides MMP9 binding proteins that specifically bind to non-mouse MMP9, such as human MMP9, Cynomolgus monkey MMP9, and rat MMP9.

The present disclosure also provides MMP9 binding proteins (e.g., anti-MMP9 antibodies and functional fragments thereof) that act as non-competitive inhibitors. A “non-competitive inhibitor” refers to an inhibitor binds at site away from substrate binding site of an enzyme, and thus can bind the enzyme and effect inhibitory activity regardless of whether or not the enzyme is bound to its substrate. Such non-competitive inhibitors can, for example, provide for a level of inhibition that can be substantially independent of substrate concentration.

MMP9 binding proteins (e.g., antibodies and functional fragments thereof) of the present disclosure include those that bind MMP9, particularly human MMP9, and having a heavy chain polypeptide (or functional fragment thereof) that has at least about 80%, 85%, 90%, 95% or more amino acid sequence identity to a heavy chain polypeptide disclosed herein. In some example, MMP9 binding proteins (e.g., antibodies and functional fragments thereof) of the present disclosure include those that bind MMP9, particularly human MMP9, and having a heavy chain polypeptide (or functional fragment thereof) that has at least about 90%, 95%, 97%, 98%, 99% or more amino acid sequence identity to a heavy chain polypeptide disclosed herein.

MMP9 binding proteins (e.g., antibodies and functional fragments thereof) of the present disclosure include those that bind MMP9, particularly human MMP9, and having a light polypeptide (or functional fragment thereof) that has at least about 80%, 85%, 90%, 95% or more amino acid sequence identity to a heavy chain polypeptide disclosed herein. MMP9 binding proteins of the present disclosure include those that bind MMP9, particularly human MMP9, and having a light chain polypeptide (or functional fragment thereof) that has at least about 80%, 85%, 90%, 95% or more amino acid sequence identity to a light chain polypeptide disclosed herein. In some example, MMP9 binding proteins of the present disclosure include those that bind MMP9, particularly human MMP9, and having a light chain polypeptide (or functional fragment thereof) that has at least about 90%, 95%, 97%, 98%, 99% or more amino acid sequence identity to a light chain polypeptide disclosed herein.

MMP9 binding proteins (e.g., antibodies and functional fragments thereof) of the present disclosure include those that bind MMP9, particularly human MMP9, and have a heavy chain polypeptide (or functional fragment thereof) having the complementarity determining regions (“CDRs”) of heavy chain polypeptide and the CDRs of a light chain polypeptide (or functional fragment thereof) as disclosed herein.

“Homology” or “identity” or “similarity” as used herein in the context of nucleic acids and polypeptides refers to the relationship between two polypeptides or two nucleic acid molecules based on an alignment of the amino acid sequences or nucleic acid sequences, respectively. Homology and identity can each be determined by comparing a position in each sequence which may be aligned for purposes of comparison. When an equivalent position in the compared sequences is occupied by the same base or amino acid, then the molecules are identical at that position; when the equivalent site occupied by the same or a similar amino acid residue (e.g., similar in steric and/or electronic nature), then the molecules can be referred to as homologous (similar) at that position. Expression as a percentage of homology/similarity or identity refers to a function of the number of identical or similar amino acids at positions shared by the compared sequences. In comparing two sequences, the absence of residues (amino acids or nucleic acids) or presence of extra residues also decreases the identity and homology/similarity.

As used herein, “identity” means the percentage of identical nucleotide or amino acid residues at corresponding positions in two or more sequences when the sequences are aligned to maximize sequence matching, i.e., taking into account gaps and insertions. Sequences are generally aligned for maximum correspondence over a designated region, e.g., a region at least about 20, 25, 30, 35, 40, 45, 50, 55, 60, 65 or more amino acids or nucleotides in length, and can be up to the full-length of the reference amino acid or nucleotide. For sequence comparison, typically one sequence acts as a reference sequence, to which test sequences are compared. When using a sequence comparison algorithm, test and reference sequences are input into a computer program, subsequence coordinates are designated, if necessary, and sequence algorithm program parameters are designated. The sequence comparison algorithm then calculates the percent sequence identity for the test sequence(s) relative to the reference sequence, based on the designated program parameters.

Examples of algorithms that are suitable for determining percent sequence identity are the BLAST and BLAST 2.0 algorithms, which are described in Altschul et al. (1990) J. Mol. Biol. 215: 403-410 and Altschul et al. (1977) Nucleic Acids Res. 25: 3389-3402, respectively. Software for performing BLAST analyses is publicly available through the National Center for Biotechnology Information (www.ncbi.nlm.nih.gov). Further exemplary algorithms include ClustalW (Higgins D., et al. (1994) Nucleic Acids Res 22: 4673-4680), available at www.ebi.ac.uk/Tools/clustalw/index.html.

Residue positions which are not identical can differ by conservative amino acid substitutions. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine.

Sequence identity between two nucleic acids can also be described in terms of hybridization of two molecules to each other under stringent conditions. The hybridization conditions are selected following standard methods in the art (see, for example, Sambrook, et al., Molecular Cloning: A Laboratory Manual, Second Edition, (1989) Cold Spring Harbor, N.Y.). An example of stringent hybridization conditions is hybridization at 50° C. or higher and 0.1×SSC (15 mM sodium chloride/1.5 mM sodium citrate). Another example of stringent hybridization conditions is overnight incubation at 42° C. in a solution: 50% formamide, 5×SSC (150 mM NaCl, 15 mM trisodium citrate), 50 mM sodium phosphate (pH7.6), 5×Denhardt's solution, 10% dextran sulfate, and 20 mg/ml denatured, sheared salmon sperm DNA, followed by washing the filters in 0.1×SSC at about 65° C. Stringent hybridization conditions are hybridization conditions that are at least as stringent as the above representative conditions, where conditions are considered to be at least as stringent if they are at least about 80% as stringent, typically at least 90% as stringent as the above specific stringent conditions.

Accordingly, the present disclosure provides, for example, antibodies or antigen binding fragments thereof, comprising a heavy chain variable region polypeptide having at least 80%, 85%, 90%, 95%, 96%, 97%, 98%, 99% or greater amino acid sequence identity to an amino acid sequence of a heavy chain variable region described herein (e.g., SEQ ID NOS:1 or 5-8), and a variable light chain polypeptide having at least 80%, 85%, 90%, 95%, 96%, 97%, 98%, 99% or greater amino acid sequence identity to an amino acid sequence of a light chain polypeptide as set forth herein (e.g., SEQ ID NOS:2 or 9-12). In one embodiment, the present disclosure provides antibodies or antigen binding fragments thereof comprising a heavy chain variable region polypeptide having at least about 95%, 96%, 97%, 98%, 99% or greater amino acid sequence identity to an amino acid sequence of a heavy chain variable region as set forth in SEQ ID NO: 7, and a variable light chain polypeptide having at least about 95%, 96%, 97%, 98%, 99% or greater amino acid sequence identity to an amino acid sequence of a light chain polypeptide as set forth in SEQ ID NO: 12. In further examples, the present disclosure provides antibodies or antigen binding fragments thereof comprising a heavy chain variable region polypeptide having at least 80%, 85%, 90%, 95%, 96%, 97%, 98%, 99% or greater amino acid sequence identity to an amino acid sequence of a heavy chain variable region as set forth in SEQ ID NOS: 32, 40, or 47, and a variable light chain polypeptide having at least 80%, 85%, 90%, 95%, 96%, 97%, 98%, 99% or greater amino acid sequence identity to an amino acid sequence of a light chain polypeptide as set forth in SEQ ID NOS: 33, 41, or 48. In some embodiment, the present disclosure provides antibodies or antigen binding fragments thereof comprising a heavy chain variable region polypeptide having at least about 95%, 96%, 97%, 98%, 99% or greater amino acid sequence identity to an amino acid sequence of a heavy chain variable region as set forth in SEQ ID NOS: 32, 40, or 47, and a variable light chain polypeptide having at least about 95%, 96%, 97%, 98%, 99% or greater amino acid sequence identity to an amino acid sequence of a light chain polypeptide as set forth in SEQ ID NOS: 33, 41, or 48. In further embodiment, the present application provides the antibodies or antigen binding fragment thereof that may compete for binding to a protein or antibody comprising an amino acid sequence having at least about 95%, 96%, 97%, 98%, 99% or greater identity to an amino acid sequence as set forth in SEQ ID NO: 7, 12, 13, 14, 15, 16, 17, or 18.

Examples of anti-MMP9 antibodies of the present disclosure are described in more detail below.

Antibodies

The MMP9 binding proteins include antibodies and functional fragments thereof, such as those that specifically bind to MMP9. As used herein, the term “antibody” means an isolated or recombinant polypeptide binding agent that comprises peptide sequences (e.g., variable region sequences) that specifically bind an antigenic epitope. The term is used in its broadest sense and specifically covers monoclonal antibodies (including full-length monoclonal antibodies), polyclonal antibodies, human antibodies, humanized antibodies, chimeric antibodies, nanobodies, diabodies, multispecific antibodies (e.g., bispecific antibodies), and antibody fragments including but not limited to Fv, scFv, Fab, Fab′ F(ab′)₂ and Fab₂, so long as they exhibit the desired biological activity. The term “human antibody” refers to antibodies containing sequences of human origin, except for possible non-human CDR regions, and does not imply that the full structure of an immunoglobulin molecule be present, only that the antibody has minimal immunogenic effect in a human (i.e., does not induce the production of antibodies to itself).

An “antibody fragment” comprises a portion of a full-length antibody, for example, the antigen binding or variable region of a full-length antibody. Such antibody fragments may also be referred to herein as “functional fragments: or “antigen binding fragments”. Examples of antibody fragments include Fab, Fab′, F(ab′)₂, and Fv fragments; diabodies; linear antibodies (Zapata et al. (1995) Protein Eng. 8(10):1057-1062); single-chain antibody molecules; and multispecific antibodies formed from antibody fragments. Papain digestion of antibodies produces two identical antigen binding fragments, called “Fab” fragments, each with a single antigen binding site, and a residual “Fc” fragment, a designation reflecting the ability to crystallize readily. Pepsin treatment yields an F(ab′)₂ fragment that has two antigen combining sites and is still capable of cross-linking antigen.

“Fv” is a minimum antibody fragment containing a complete antigen-recognition and -binding site. This region consists of a dimer of one heavy- and one light-chain variable domain in tight, non-covalent association. It is in this configuration that the three complementarity-determining regions (CDRs) of each variable domain interact to define an antigen binding site on the surface of the V_{H}-V_{L} dimer. Collectively, the six CDRs confer antigen binding specificity to the antibody. However, even a single variable domain (or an isolated V_{H} or V_{L} region comprising only three of the six CDRs specific for an antigen) has the ability to recognize and bind antigen, although generally at a lower affinity than does the entire Fᵥ fragment.

The “F_{ab}” fragment also contains, in addition to heavy and light chain variable regions, the constant domain of the light chain and the first constant domain (CH₁) of the heavy chain. Fab fragments were originally observed following papain digestion of an antibody. Fab′ fragments differ from Fab fragments in that F(ab′) fragments contain several additional residues at the carboxy terminus of the heavy chain CH₁ domain, including one or more cysteines from the antibody hinge region. F(ab′)₂ fragments contain two Fab fragments joined, near the hinge region, by disulfide bonds, and were originally observed following pepsin digestion of an antibody. Fab′-SH is the designation herein for Fab′ fragments in which the cysteine residue(s) of the constant domains bear a free thiol group. Other chemical couplings of antibody fragments are also known.

The “light chains” of antibodies (immunoglobulins) from any vertebrate species can be assigned to one of two clearly distinct types, called kappa and lambda, based on the amino acid sequences of their constant domains. Depending on the amino acid sequence of the constant domain of their heavy chains, immunoglobulins can be assigned to five major classes: IgA, IgD, IgE, IgG, and IgM, and several of these may be further divided into subclasses (isotypes), e.g., IgG1, IgG2, IgG3, IgG4, IgA1, and IgA2.

“Single-chain Fv” or “sFv” or “scFv” antibody fragments comprise the V_{H} and V_{L} domains of antibody, wherein these domains are present in a single polypeptide chain. In some embodiments, the Fv polypeptide further comprises a polypeptide linker between the V_{H} and V_{L} domains, which enables the sFv to form the desired structure for antigen binding. For a review of sFv, see Pluckthun, in The Pharmacology of Monoclonal Antibodies, vol. 113 (Rosenburg and Moore eds.) Springer-Verlag, New York, pp. 269-315 (1994).

The term “diabodies” refers to small antibody fragments with two antigen binding sites, which fragments comprise a heavy-chain variable domain (V_{H}) connected to a light-chain variable domain (V_{L}) in the same polypeptide chain (V_{H}-V_{L}). By using a linker that is too short to allow pairing between the two domains on the same chain, the domains are forced to pair with the complementary domains of another chain, thereby creating two antigen binding sites. Diabodies are additionally described, for example, in EP 404,097; WO 93/11161 and Hollinger et al. (1993) Proc. Natl. Acad. Sci. USA 90:6444-6448.

An “isolated” antibody is one that has been identified and separated and/or recovered from a component of its natural environment. Components of its natural environment may include enzymes, hormones, and other proteinaceous or nonproteinaceous solutes. In some embodiments, an isolated antibody is purified (1) to greater than 95% by weight of antibody as determined by the Lowry method, for example, more than 99% by weight, (2) to a degree sufficient to obtain at least 15 residues of N-terminal or internal amino acid sequence, e.g., by use of a spinning cup sequenator, or (3) to homogeneity by gel electrophoresis (e.g., SDS-PAGE) under reducing or nonreducing conditions, with detection by Coomassie blue or silver stain. The term “isolated antibody” includes an antibody in situ within recombinant cells, since at least one component of the antibody's natural environment will not be present. In certain embodiments, isolated antibody is prepared by at least one purification step.

As used herein, “immunoreactive” refers to antibodies or fragments thereof that are specific to a sequence of amino acid residues (“binding site” or “epitope”), yet if are cross-reactive to other peptides/proteins, are not toxic at the levels at which they are formulated for administration to human use. “Epitope” refers to that portion of an antigen capable of forming a binding interaction with an antibody or antigen binding fragment thereof. An epitope can be a linear peptide sequence (i.e., “continuous”) or can be composed of noncontiguous amino acid sequences (i.e., “conformational” or “discontinuous”). The term “preferentially binds” means that the binding agent binds to the binding site with greater affinity than it binds unrelated amino acid sequences.

Anti-MMP9 antibodies can be described in terms of the CDRs of the heavy and light chains. As used herein, the term “CDR” or “complementarity determining region” is intended to mean the non-contiguous antigen combining sites found within the variable region of both heavy and light chain polypeptides. These particular regions have been described by Kabat et al., J. Biol. Chem. 252:6609-6616 (1977); Kabat et al., U.S. Dept. of Health and Human Services, “Sequences of proteins of immunological interest” (1991); by Chothia et al., J. Mol. Biol. 196:901-917 (1987); and MacCallum et al., J. Mol. Biol. 262:732-745 (1996), where the definitions include overlapping or subsets of amino acid residues when compared against each other. Nevertheless, application of either definition to refer to a CDR of an antibody or grafted antibodies or variants thereof is intended to be within the scope of the term as defined and used herein. The amino acid residues which encompass the CDRs as defined by each of the above cited references are set forth below in Table 1A as a comparison.

TABLE 1A
CDR Definitions

| | Kabat¹ | Chothia² | MacCallum³ |
|---|---|---|---|
| V_{H} CDR1 | 31-35 | 26-32 | 30-35 |
| V_{H} CDR2 | 50-65 | 53-55 | 47-58 |
| V_{H} CDR3 | 95-102 | 96-101 | 93-101 |
| V_{L} CDR1 | 24-34 | 26-32 | 30-36 |
| V_{L} CDR2 | 50-56 | 50-52 | 46-55 |
| V_{L} CDR3 | 89-97 | 91-96 | 89-96 |

¹Residue numbering follows the nomenclature of Kabat et al., supra
²Residue numbering follows the nomenclature of Chothia et al., supra
³Residue numbering follows the nomenclature of MacCallum et al., supra

As used herein, the term “framework” when used in reference to an antibody variable region is intended to mean all amino acid residues outside the CDR regions within the variable region of an antibody. A variable region framework is generally a discontinuous amino acid sequence between about 100-120 amino acids in length but is intended to reference only those amino acids outside of the CDRs. As used herein, the term “framework region” is intended to mean each domain of the framework that is separated by the CDRs.

In some embodiments, an antibody is a humanized antibody or a human antibody. Humanized antibodies include human immunoglobulins (recipient antibody) in which residues from a complementary-determining region (CDR) of the recipient are replaced by residues from a CDR of a non-human species (donor antibody) such as mouse, rat or rabbit having the desired specificity, affinity and capacity. Thus, humanized forms of non-human (e.g., murine) antibodies are chimeric immunoglobulins which contain minimal sequence derived from non-human immunoglobulin. The non-human sequences are located primarily in the variable regions, particularly in the complementarity-determining regions (CDRs). In some embodiments, Fv framework residues of the human immunoglobulin are replaced by corresponding non-human residues. Humanized antibodies can also comprise residues that are found neither in the recipient antibody nor in the imported CDR or framework sequences. In certain embodiments, a humanized antibody comprises substantially all of at least one, and typically two, variable domains, in which all or substantially all of the CDRs correspond to those of a non-human immunoglobulin and all or substantially all of the framework regions are those of a human immunoglobulin consensus sequence. For the purposes of the present disclosure, humanized antibodies can also include immunoglobulin fragments, such as Fv, Fab, Fab′, F(ab′)₂ or other antigen binding subsequences of antibodies.

The humanized antibody can also comprise at least a portion of an immunoglobulin constant region (Fc), typically that of a human immunoglobulin. See, for example, Jones et al. (1986) Nature 321:522-525; Riechmann et al. (1988) Nature 332:323-329; and Presta (1992) Curr. Op. Struct. Biol. 2:593-596.

Methods for humanizing non-human antibodies are known in the art. Generally, a humanized antibody has one or more amino acid residues introduced into it from a source that is non-human. These non-human amino acid residues are often referred to as “import” or “donor” residues, which are typically obtained from an “import” or “donor” variable domain. For example, humanization can be performed essentially according to the method of Winter and co-workers, by substituting rodent CDRs or CDR sequences for the corresponding sequences of a human antibody. See, for example, Jones et al., supra; Riechmann et al., supra and Verhoeyen et al. (1988) Science 239:1534-1536. Accordingly, such “humanized” antibodies include chimeric antibodies (U.S. Pat. No. 4,816,567), wherein substantially less than an intact human variable domain has been substituted by the corresponding sequence from a non-human species. In certain embodiments, humanized antibodies are human antibodies in which some CDR residues and optionally some framework region residues are substituted by residues from analogous sites in rodent antibodies (e.g., murine monoclonal antibodies).

Human antibodies can also be produced, for example, by using phage display libraries. Hoogenboom et al. (1991) J. Mol. Biol, 227:381; Marks et al. (1991) J. Mol. Biol. 222:581. Other methods for preparing human monoclonal antibodies are described by Cole et al. (1985) “Monoclonal Antibodies and Cancer Therapy,” Alan R. Liss, p. 77 and Boerner et al. (1991) J. Immunol. 147:86-95.

Human antibodies can be made by introducing human immunoglobulin loci into transgenic animals (e.g., mice) in which the endogenous immunoglobulin genes have been partially or completely inactivated. Upon immunological challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al. (1992) Bio/Technology 10:779-783 (1992); Lonberg et al. (1994) Nature 368: 856-859; Morrison (1994) Nature 368:812-813; Fishwald et al. (1996) Nature Biotechnology 14:845-851; Neuberger (1996) Nature Biotechnology 14:826; and Lonberg et al. (1995) Intern. Rev. Immunol. 13:65-93.

Antibodies can be affinity matured using known selection and/or mutagenesis methods as described above. In some embodiments, affinity matured antibodies have an affinity which is five times or more, ten times or more, twenty times or more, or thirty times or more than that of the starting antibody (generally murine, rabbit, chicken, humanized or human) from which the matured antibody is prepared.

An antibody can also be a bispecific antibody. Bispecific antibodies are monoclonal, and may be human or humanized antibodies that have binding specificities for at least two different antigens. In the present case, the two different binding specificities can be directed to two different MMPs, or to two different epitopes on a single MMP (e.g., MMP9).

An antibody as disclosed herein can also be an immunoconjugate. Such immunoconjugates comprise an antibody (e.g., to MMP9) conjugated to a second molecule, such as a reporter An immunoconjugate can also comprise an antibody conjugated to a cytotoxic agent such as a chemotherapeutic agent, a toxin (e.g., an enzymatically active toxin of bacterial, fungal, plant, or animal origin, or fragments thereof), or a radioactive isotope (i.e., a radioconjugate).

An antibody that “specifically binds to” or is “specific for” a particular polypeptide or an epitope refers to the selective binding of the antibody to the target antigen or epitope; these terms, and methods for determining specific binding, are well understood in the art. An antibody exhibits “specific binding” for a particular target antigen or epitope if it binds with greater affinity, avidity, more readily, and/or with greater duration to that target antigen or epitope than it does with other substances. In some embodiments, the antibody that specifically binds to the polypeptide or epitope is one that that binds to that particular polypeptide or epitope without substantially binding to any other polypeptide or polypeptide epitope. In some embodiments, the provided antibodies specifically bind to human MMP9 with a dissociation constant (K_{d}) equal to or lower than 100 nM, optionally lower than 10 nM, optionally lower than 1 nM, optionally lower than 0.5 nM, optionally lower than 0.1 nM, optionally lower than 0.01 nM, or optionally lower than 0.005 nM, in certain examples, between 0.1 and 0.2 nM, or between 0.1 and 10 pM, e.g., between 0.4 and 9 pm, such as between 0.4 and 8.8 pm, in the form of monoclonal antibody, scFv, Fab, or other form of antibody measured at a temperature of about 4° C., 25° C., 37° C. or 42° C.

In certain embodiments, an antibody of the present disclosure binds to one or more processing sites (e.g., sites of proteolytic cleavage) in MMP9, thereby effectively blocking processing of the proenzyme or preproenzyme to the catalytically active enzyme, and thus reducing the proteolytic activity of the MMP9.

In certain embodiments, an antibody according to the present disclosure binds to MMP9 with an affinity at least 2 times, at least 5 times, at least 10 times, at least 25 times, at least 50 times, at least 100 times, at least 500 times, or at least 1000 times greater than its binding affinity for another MMP. Binding affinity can be measured by any method known in the art and can be expressed as, for example, on-rate, off-rate, dissociation constant (K_{d}), equilibrium constant (K_{eq}) or any term in the art.

In certain embodiments, an antibody according to the present disclosure is one that inhibits the enzymatic (i.e., catalytic) activity of MMP9, such as a non-competitive inhibitor of the catalytic activity of MMP9. In certain embodiments, an antibody according to the present disclosure binds within the catalytic domain of MMP9. In additional embodiments, an antibody according to the present disclosure binds outside the catalytic domain of MMP9.

Also provided are antibodies or antigen binding fragments thereof that compete with any one or more of the anti-MMP9 antibodies or antigen binding fragments thereof described herein for binding to MMP9. Thus, the present disclosure contemplates anti-MMP9 antibodies, and functional fragments thereof, that compete for binding with, for example, an antibody having a heavy chain polypeptide of any of SEQ ID NOS: 1 or 5-8, a light chain polypeptide of SEQ ID NOS: 2 or 9-12, or combinations thereof. In one embodiment, the anti-MMP9 antibody, or functional fragment thereof, competes for binding to human MMP9 with the antibody described herein as AB0041. In some embodiment, the anti-MMP9 antibody or functional fragment thereof competes for binding to human MMP9 with the antibody described herein as AB0045. In certain embodiment, the anti-MMP9 antibody or functional fragment thereof competes for binding to human MMP9 with the antibody described herein as AB0046. In additional embodiment, the anti-MMP9 antibody or functional fragment thereof competes for binding to human MMP9 with the antibody described herein as M4. In other embodiment, the anti-MMP9 antibody or functional fragment thereof competes for binding to human MMP9 with the antibody described herein as M12.

Also provided are antibodies and fragments thereof that bind to the same epitope, e.g., MMP9 epitope as any one or more of the antibodies described herein. Also provided are antibodies and fragments that specifically bind to an epitope of MMP9, where the epitope includes an amino acid residue within a particular region of MMP9 or multiple regions of MMP9. Further provided are anti-MMP9 antibody or antigen binding fragment thereof that compete for binding to a protein or antibody that binds to the epitope or region described herein. Such regions can include, for example, structural loops and/or other structural domains of MMP9, such as those shown to be important for binding to exemplary antibodies described herein. Typically, the regions are defined according to amino acid residue positions on the full-length MMP9 sequence, e.g., SEQ ID NO: 27. In some example, the epitope contains an amino acid residue 104-202 of SEQ ID NO: 27. In one example, the epitope contains an amino acid residue (i.e., one or more amino acid residue(s)) within a region that is residues 104-119 residues 159-166, or residues 191-202 of SEQ ID NO: 27. In some aspects, the epitope includes an amino acid residue (i.e., one or more amino acid residue(s)) within a region of MMP9 that is residues 104-119 of SEQ ID NO: 27, an amino acid residue within a region of MMP9 that is residues 159-166 of SEQ ID NO: 27, and an amino acid residue within a region of MMP9 that is residues 191-202 of SEQ ID NO: 27. In some cases, the epitope includes E111, D113, R162, or I198 of SEQ ID NO: 27. In some cases, it includes R162 of SEQ ID NO: 27. In some cases, it includes E111, D113, R162, and I198 of SEQ ID NO: 27.

MMP9 Sequence

The amino acid sequence of human MMP9 protein is as follows:

| (SEQ ID NO: 27) | |
|---|---|
| MSLWQPLVLV LLVLGCCFAA PRQRQSTLVL FPGDLRTNLT DRQLAEEYLY | 50 |
| RYGYTRVAEM RGESKSLGPA LLLLQKQLSL PETGELDSAT LKAMRTPRCG | 100 |
| VPDLGRFQTF EGDLKWHHHN ITYWIQNYSE DLPRAVIDDA FARAFALWSA | 150 |
| VTPLTFTRVY SRDADIVIQF GVAEHGDGYP FDGKDGLLAH AFPPGPGIQG | 200 |
| DAHFDDDELW SLGKGVVVPT RFGNADGAAC HFPFIFEGRS YSACTTDGRS | 250 |
| DGLPWCSTTA NYDTDDRFGF CPSERLYTRD GNADGKPCQF PFIFQGQSYS | 300 |
| ACTTDGRSDG YRWCATTANY DRDKLFGFCP TRADSTVMGG NSAGELCVFP | 350 |
| FTFLGKEYST CTSEGRGDGR LWCATTSNFD SDKKWGFCPD QGYSLFLVAA | 400 |
| HEFGHALGLD HSSVPEALMY PMYRFTEGPP LHKDDVNGIR HLYGPRPEPE | 450 |
| PRPPTTTTPQ PTAPPTVCPT GPPTVHPSER PTAGPTGPPS AGPTGPPTAG | 500 |
| PSTATTVPLS PVDDACNVNI FDAIAEIGNQ LYLFKDGKYW RFSEGRGSRP | 550 |
| QGPFLIADKW PALPRKLDSV FEEPLSKKLF FFSGRQVWVY TGASVLGPRR | 600 |
| LDKLGLGADV AQVTGALRSG RGKMLLFSGR RLWRFDVKAQ MVDPRSASEV | 650 |
| DRMFPGVPLD THDVFQYREK AYFCQDRFYW RVSSRSELNQ VDQVGYVTYD | 700 |
| ILQCPED | |

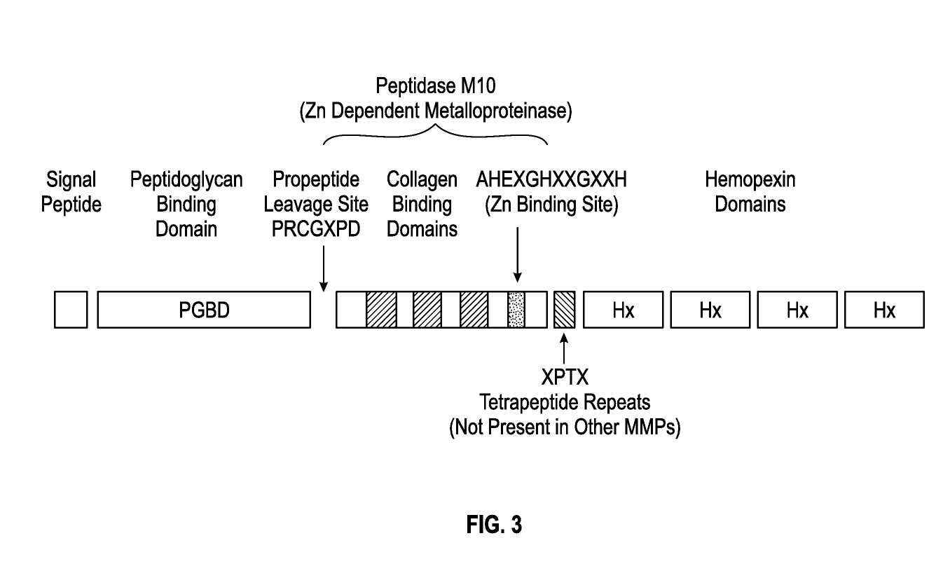

Protein domains are shown schematically in FIG. 3 and are indicated below:

| Amino Acid # | Feature |
|---|---|
| 1-19 | Signal Peptide |
| 38-98 | Peptidoglycan Binding Domain |
| R98/C99 | Cysteine-switch active pocket |
| 112-445 | Zn dependent metalloproteinase domain |
| 223-271 | Fibronectin type II domain (gelatin binding domain) |
| 281-329 | Fibronectin type II domain (gelatin binding domain) |
| 340-388 | Fibronectin type II domain (gelatin binding domain) |
| 400-411 | Zn binding region |
| 521-565 | Hemopexin-like domain |
| 567-608 | Hemopexin-like domain |
| 613-659 | Hemopexin-like domain |
| 661-704 | Hemopexin-like domain |

The amino acid sequence of mature full-length human MMP9 (which is the amino acid sequence of the propolypeptide of SEQ ID NO:27 without the signal peptide) is:

(SEQ ID NO: 28)
APRQRQSTLVL FPGDLRTNLT DRQLAEEYLY RYGYTRVAEM
RGESKSLGPA LLLLQKQLSL PETGELDSAT LKAMRTPRCG
VPDLGRFQTF EGDLKWHHHN ITYWIQNYSE DLPRAVIDDA
FARAFALWSA VTPLTFTRVY SRDADIVIQF GVAEHGDGYP
FDGKDGLLAH AFPPGPGIQG DAHFDDDELW SLGKGVVVPT
RFGNADGAAC HFPFIFEGRS YSACTTDGRS DGLPWCSTTA
NYDTDDRFGF CPSERLYTRD GNADGKPCQF PFIFQGQSYS
ACTTDGRSDG YRWCATTANY DRDKLFGFCP TRADSTVMGG
NSAGELCVFP FTFLGKEYST CTSEGRGDGR LWCATTSNFD
SDKKWGFCPD QGYSLFLVAA HEFGHALGLD HSSVPEALMY
PMYRFTEGPP LHKDDVNGIR HLYGPRPEPE PRPPTTTTPQ
PTAPPTVCPT GPPTVHPSER PTAGPTGPPS AGPTGPPTAG
PSTATTVPLS PVDDACNVNI FDAIAEIGNQ LYLFKDGKYW
RFSEGRGSRP QGPFLIADKW PALPRKLDSV FEEPLSKKLF
FFSGRQVWVY TGASVLGPRR LDKLGLGADV AQVTGALRSG
RGKMLLFSGR RLWRFDVKAQ MVDPRSASEV DRMFPGVPLD
THDVFQYREK AYFCQDRFYW RVSSRSELNQ VDQVGYVTYD
ILQCPED

The amino acid sequence of the signal peptide is MSLWQPLVLVLLVLGCCFA (SEQ ID NO:29).

Also provided are MMP9 polypeptides, including mutant MMP9 polypeptides. Such peptides are useful, for example, in generating and selecting antibodies and fragments as provided herein. Exemplary polypeptides include those having an amino acid sequence containing residues 111-198 of SEQ ID NO: 27, and those having an amino acid sequence containing residues 111-198 of SEQ ID NO: 27 with an amino acid substitution at residue 111, 113, 162, or 198 of SEQ ID NO 27 or with an amino acid substitution at all such residues. Such polypeptides find use, for example, in selecting antibodies that bind to epitopes containing such residues and/or for which such residues of MMP9 are important for binding, such as those described herein.

The present disclosure contemplates MMP9 binding proteins that bind any portion of MMP9, e.g., human MMP9, with MMP9 binding proteins that preferentially bind MMP9 relative to other MMPs being of particular interest.

Anti-MMP9 antibodies, and functional fragments thereof, can be generated accordingly to methods well known in the art. Exemplary anti-MMP9 antibodies are provided below.

Mouse Monoclonal Anti-MMP9 Antibodies

A mouse monoclonal antibody to human MMP9 was obtained as. This antibody contains a mouse IgG2b heavy chain and a mouse kappa light chain, and is denoted AB0041.

The amino acid sequence of the AB0041 heavy chain is as follows:

(SEQ ID NO: 1)
MAVLVLFLCLVAFPSCVLSQVQLKESGPGLVAPSQSLSITCTVSGFSLLS
YGVHWVRQPPGKGLEWLGVIWTGGTTNYNSALMSRLSISKDDSKSQVFLK
MNSLQTDDTAIYYCARYYYGMDYWGQGTSVTVSSAKTTPPSVYPLAPGCG
DTTGSSVTLGCLVKGYFPESVTVTWNSGSLSSSVHTFPALLQSGLYTMSS
SVTVPSSTWPSQTVTCSVAHPASSTTVDKKLEPSGPISTINPCPPCKECH
KCPAPNLEGGPSVFIFPPNIKDVLMISLTPKVTCVVVDVSEDDPDVRISW
FVNNVEVHTAQTQTHREDYNSTIRVVSALPIQHQDWMSGKEFKCKVNNKD
LPSPIERTISKIKGLVRAPQVYILPPPAEQLSRKDVSLTCLVVGFNPGDI
SVEWTSNGHTEENYKDTAPVLDSDGSYFIYSKLDIKTSKWEKTDSFSCNV
RHEGLKNYYLKKTISRSPGK

The signal sequence is underlined, and the sequence of the IgG2b constant region is presented italics. Without the signal peptide, the heavy chain of the AB0041 antibody has the sequence set forth in SEQ ID NO: 58

(QVQLKESGPGLVAPSQSLSITCTVSGFSLLSYGVHWVRQPPGKGLEWLG
VIWTGGTTNYNSALMSRLSISKDDSKSQVFLKMNSLQTDDTAIYYCARYY
YGMDYWGQGTSVTVSSAKTTPPSVYPLAPGCGDTTGSSVTLGCLVKGYFP
ESVTVTWNSGSLSSSVHTFPALLQSGLYTMSSSVTVPSSTWPSQTVTCSV
AHPASSTTVDKKLEPSGPISTINPCPPCKECHKCPAPNLEGGPSVFIFPP
NIKDVLMISLTPKVTCVVVDVSEDDPDVRISWFVNNVEVHTAQTQTHRED
YNSTIRVVSALPIQHQDWMSGKEFKCKVNNKDLPSPIERTISKIKGLVRA
PQVYILPPPAEQLSRKDVSLTCLVVGFNPGDISVEWTSNGHTEENYKDTA
PVLDSDGSYFIYSKLDIKTSKWEKTDSFSCNVRHEGLKNYYLKKTISRSP
GK).

The amino acid sequence of the AB0041 light chain is as follows:

(SEQ ID NO: 2)
MESQIQVFVFVFLWLSGVDGDIVMTQSHKFMSTSVGDRVSITCKASQDVR
NTVAWYQQKTGQSPKLLIYSSSYRNTGVPDRFTGSGSGTDFTFTISSVQA
EDLAVYFCQQHYITPYTFGGGTKLEIKRADAAPTVSIFPPSSEQLTSGGA
SVVCFLNNFYPKDINVKWKIDGSERQNGVLNSWTDQDSKDSTYSMSSTLT
LTKDEYERHNSYTCEATHKTSTSPIVKSFNRNEC

The signal sequence is underlined, and the sequence of the kappa constant region is presented in italics. Without the signal peptide, the light chain of the AB0041 antibody has the sequence set forth in SEQ ID NO: 59

(DIVMTQSHKFMSTSVGDRVSITCKASQDVRNTVAWYQQKTGQSPKLLIY
SSSYRNTGVPDRFTGSGSGTDFTFTISSVQAEDLAVYFCQQHYITPYTFG
GGTKLEIKRADAAPTVSIFPPSSEQLTSGGASVVCFLNNFYPKDINVKWK
IDGSERQNGVLNSWTDQDSKDSTYSMSSTLTLTKDEYERHNSYTCEATHK
TSTSPIVKSFNRNEC).

The following amino acid sequence comprises the framework regions and complementarity-determining regions (CDRs) of the variable region of the IgG2b heavy chain of AB0041 (with CDRs underlined):

(SEQ ID NO: 3)
QVQLKESGPGLVAPSQSLSITCTVSGFSLLSYGVHWVRQPPGKGLEWLGV
IWTGGTTNYNSALMSRLSISKDDSKSQVFLKMNSLQTDDTAIYYCARYYY
GMDYWGQGTSVTVSS

The following amino acid sequence comprises the framework regions and complementarity-determining regions (CDRs) of the variable region of the kappa light chain of AB0041 (with CDRs underlined):

(SEQ ID NO: 4)
DIVMTQSHKFMSTSVGDRVSITCKASQDVRNTVAWYQQKTGQSPKLLIYS
SSYRNTGVPDRFTGSGSGTDFTFTISSVQAEDLAVYFCQQHYITPYTFGG
GTKLEIK

Other exemplary mouse anti-human MMP9 antibodies (e.g., M4 and M12) are described herein. An exemplary anti-mouse MMP9 antibody (AB0046) is described herein. In some embodiments, provided are uses of such anti-mouse antibodies as surrogate antibodies for testing and assessing the MMP9-inhibition methods, e.g., therapeutic methods, as provided herein.

Heavy-Chain Variants

The amino acid sequences of the variable regions of the AB0041 heavy and light chains were separately modified, by altering framework region sequences in the heavy and light chain variable regions. The effect of these sequence alterations was to deplete the antibody of human T-cell epitopes, thereby reducing or abolishing its immunogenicity in humans (Antitope, Babraham, UK).

Four heavy-chain variants were constructed, in a human IgG4 heavy chain background containing a S241P amino acid change that stabilizes the hinge domain (Angal et al. (1993) Molec. Immunol. 30:105-108), and are denoted VH1, VH2, VH3 and VH4. The amino acid sequences of their framework regions and CDRs are as follows:

VH1

(SEQ ID NO: 5)
QVQLQESGPGLVKPSETLSLTCTVSGFSLLSYGVHWVRQPPGKGLEWLGV
IWTGGTTNYNSALMSRLTISKDDSKSTVYLKMNSLKTEDTAIYYCARYYY
GMDYWGQGTSVTVSS

VH2

(SEQ ID NO: 6)
QVQLQESGPGLVKPSETLSLTCTVSGFSLLSYGVHWVRQPPGKGLEWLGV
IWTGGTTNYNSALMSRLTISKDDSKNTVYLKMNSLKTEDTAIYYCARYYY
GMDYWGQGTLVTVSS

VH3

(SEQ ID NO: 7)
QVQLQESGPGLVKPSETLSLTCTVSGFSLLSYGVHWVRQPPGKGLEWLGV
IWTGGTTNYNSALMSRFTISKDDSKNTVYLKMNSLKTEDTAIYYCARYYY
GMDYWGQGTLVTVSS

VH4

(SEQ ID NO: 8)
QVQLQESGPGLVKPSETLSLTCTVSGFSLLSYGVHWVRQPPGKGLEWLGV
IWTGGTTNYNSALMSRFTISKDDSKNTLYLKMNSLKTEDTAIYYCARYYY
GMDYWGQGTLVTVSS

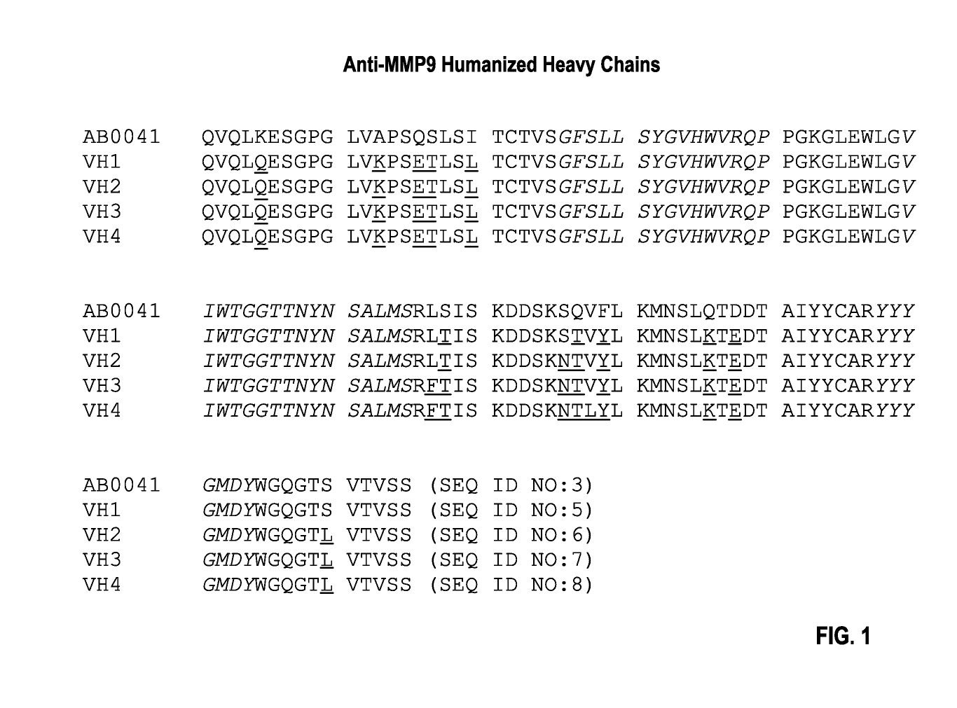

FIG. 1 shows an alignment of the amino acid sequences of the variable regions of the humanized heavy chains and indicates the differences in amino acid sequences in the framework regions among the four variants.

Light-Chain Variants

Four light-chain variants were constructed, in a human kappa chain background, and are denoted Vk1, Vk2, Vk3 and Vk4. The amino acid sequences of their framework regions and CDRs are as follows:

Vk1

(SEQ ID NO: 9)
DIVMTQSPSFLSASVGDRVTITCKASQDVRNTVAWYQQKTGKAPKLLIYS
SSYRNTGVPDRFTGSGSGTDFTLTISSLQAEDVAVYFCQQHYITPYTFGG
GTKVEIK

Vk2

(SEQ ID NO: 10)
DIVMTQSPSSLSASVGDRVTITCKASQDVRNTVAWYQQKPGKAPKLLIYS
SSYRNTGVPDRFTGSGSGTDFTLTISSLQAEDVAVYFCQQHYITPYTFGG
GTKVEIK

Vk3

(SEQ ID NO: 11)
DIQMTQSPSSLSASVGDRVTITCKASQDVRNTVAWYQQKPGKAPKLLIYS
SSYRNTGVPDRFSGSGSGTDFTLTISSLQAEDVAVYFCQQHYITPYTFGG
GTKVEIK

Vk4

(SEQ ID NO: 12)
DIQMTQSPSSLSASVGDRVTITCKASQDVRNTVAWYQQKPGKAPKLLIYS
SSYRNTGVPDRFSGSGSGTDFTLTISSLQAEDVAVYYCQQHYITPYTFGG
GTKVEIK

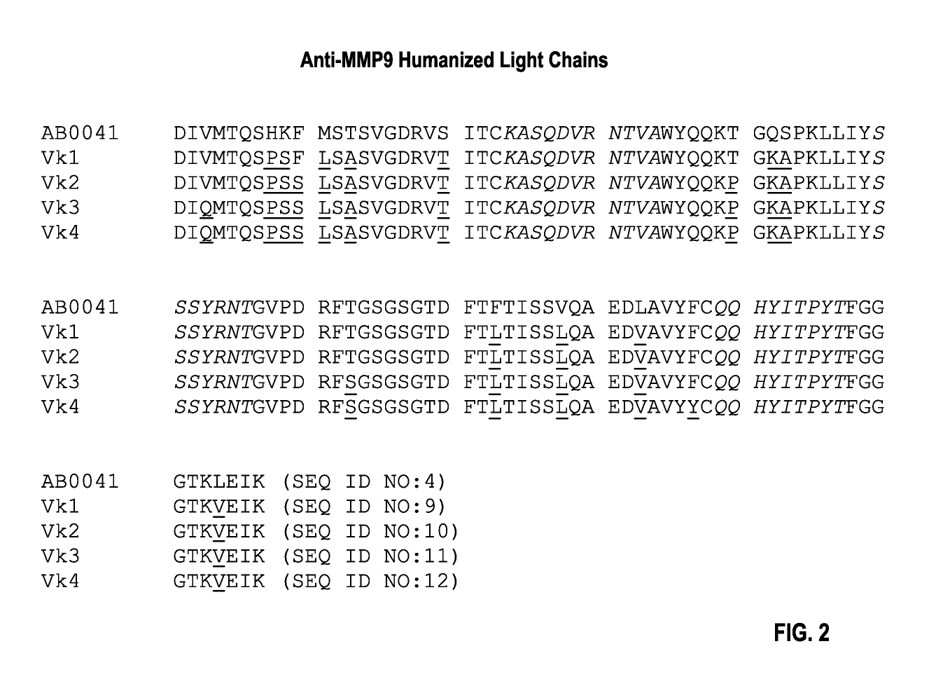

FIG. 2 shows an alignment of the amino acid sequences of the variable regions of the humanized light chains and indicates the differences in amino acid sequences in the framework regions among the four variants.

The humanized heavy and light chains are combined in all possible pair-wise combinations to generate a number of functional humanized anti-MMP9 antibodies. For example, provided are antibodies with a heavy chain variable (VH) region having the amino acid sequence set forth in any of SEQ ID NOs: 3, 5, 6, 7, and 8; antibodies having a light chain variable (VL) region having the amino acid sequence set forth in any of SEQ ID NOs: 4, 9, 10, 11, and 12; and antibodies with a heavy chain variable (VH) region having the amino acid sequence set forth in any of SEQ ID NOs: 3, 5, 6, 7, and 8 and a light chain variable (VL) region having the amino acid sequence set forth in any of SEQ ID NOs: 4, 9, 10, 11, and 12, as well as antibodies that compete for binding to MMP9 with such antibodies and antibodies having at least at or about 75%, 80%, 85%, 90%, 91%, 92%, 93%, 94%, 95%, 96%, 97%, 98%, 99% or more sequence identity with such antibodies. In one example, the antibody has a VH region with an amino acid sequence having at least at or about 75%, 80%, 85%, 90%, 91%, 92%, 93%, 94%, 95%, 96%, 97%, 98%, 99% or more sequence identity with SEQ ID NO: 7 and a VL region with an amino acid sequence having at least at or about 75%, 80%, 85%, 90%, 91%, 92%, 93%, 94%, 95%, 96%, 97%, 98%, 99% or more sequence identity with SEQ ID NO: 12, or a VH region of SEQ ID NO: 7 and a VL region of SEQ ID NO: 12. In additional example, the antibody has a VH region with an amino acid sequence having at least at or about 95%, 96%, 97%, 98%, 99% or more sequence identity with SEQ ID NO: 7. In further example, the antibody has a VL region with an amino acid sequence having at least at or about 95%, 96%, 97%, 98%, 99% or more sequence identity with SEQ ID NO: 12. In some example, the antibody has a VH region of SEQ ID NO: 7 and a VL region of SEQ ID NO: 12.

Additional heavy chain variable region amino acid sequences having 75% or more, 80% or more, 90% or more, 95% or more, or 99% or more homology to the heavy chain variable region sequences disclosed herein are also provided. Furthermore, additional light chain variable region amino acid sequences having 75% or more, 80% or more, 90% or more, 95% or more, or 99% or more homology to the light chain variable region sequences disclosed herein are also provided.

Additional heavy chain variable region amino acid sequences having 75% or more, 80% or more, 90% or more, 95% or more, or 99% or more sequence identity to the heavy chain variable region sequences disclosed herein are also provided. Furthermore, additional light chain variable region amino acid sequences having 75% or more, 80% or more, 90% or more, 95% or more, or 99% or more sequence identity to the light chain variable region sequences disclosed herein are also provided.

Complementarity-Determining Regions (CDRs)

In some embodiments, the CDRs of the heavy chain of exemplary provided anti-MMP9 antibodies as disclosed herein have the following amino acid sequences:

(SEQ ID NO: 13)
CDR1: GFSLLSYGVH
(SEQ ID NO: 14)
CDR2: VIWTGGTTNYNSALMS
(SEQ ID NO: 15)
CDR3: YYYGMDY

Thus, among the provided anti-MMP9 antibodies are antibodies having a heavy chain CDR1 region with an amino acid sequence as set forth in SEQ ID NO: 13, antibodies having a heavy chain CDR2 region with an amino acid sequence set forth in SEQ ID NO: 14, and antibodies having a heavy chain CDR3 region with an amino acid sequence as set forth in SEQ ID NO: 15, and antibodies that compete for binding with or bind to the same epitope on MMP9 as such antibodies. In some cases, the antibodies contain VH CDRs having the sequences set forth in SEQ ID NO: 15. In some cases, the antibodies contain VH CDRs having the sequences set forth in SEQ ID NOs: 13 and 14. In some cases, the antibodies contain VH CDRs having the sequences set forth in SEQ ID NOs: 13 and 15. In some cases, the antibodies contain VH CDRs having the sequences set forth in SEQ ID NOs: 14 and 15. In some cases, the antibodies contain VH CDRs having the sequences set forth in SEQ ID NOs: 13, 14, and 15.

In some embodiments, the CDRs of the light chain of exemplary anti-MMP9 antibodies as disclosed herein have the following amino acid sequences:

(SEQ ID NO: 16)
CDR1: KASQDVRNTVA
(SEQ ID NO: 17)
CDR2: SSSYRNT
(SEQ ID NO: 18)
CDR3: QQHYITPYT

Thus, among the provided anti-MMP9 antibodies are antibodies having a light chain CDR1 region with an amino acid sequence as set forth in SEQ ID NO: 16, antibodies having a light chain CDR2 region with an amino acid sequence set forth in SEQ ID NO: 17, and antibodies having a light chain CDR3 region with an amino acid sequence as set forth in SEQ ID NO: 18, and antibodies that compete for binding with or bind to the same epitope on MMP9 as such antibodies. In some cases, the antibodies contain VL CDRs having the sequences set forth in SEQ ID NO: 18. In some cases, the antibodies contain VL CDRs having the sequences set forth in SEQ ID NOs: 16 and 17. In some cases, the antibodies contain VL CDRs having the sequences set forth in SEQ ID NOs: 16 and 18. In some cases, the antibodies contain VL CDRs having the sequences set forth in SEQ ID NOs: 17 and 18. In some cases, the antibodies contain VL CDRs having the sequences set forth in SEQ ID NOs: 16, 17, and 18.

An exemplary humanized variant anti-MMP9 antibody, AB0045 (humanized, modified IgG4 (S241P)) contains the humanized AB0041 heavy chain variant VH3 (having the sequence set forth in SEQ ID NO: 7

(QVQLQESGPGLVKPSETLSLTCTVSGFSLLSYGVHWVRQPPGKGLEWLG
VIWTGGTTNYNSALMSRFTISKDDSKNTVYLKMNSLKTEDTAIYYCARYY
YGMDYWGQGTLVTVSS)

and the humanized AB0041 light chain variant VH4 (having the light chain sequence set forth in Vk4 (having the sequence set forth in SEQ ID NO: 12

(DIQMTQSPSSLSASVGDRVTITCKASQDVRNTVAWYQQKPGKAPKLLIY
SSSYRNTGVPDRFSGSGSGTDFTLTISSLQAEDVAVYYCQQHYITPYTFG
GGTKVEIK)).

The AB0045 antibody contains 1312 amino acids in length, is composed of two heavy chains and two light chains, and has a theoretical pI of about 7.90, extinction coefficient of about 1.50 AU/cm at 280 nm for 1 g/L, a molecular weight of about 144 kDa, and density of about 1 g/mL in formulation buffer (50-100 mg/mL product concentration).

The heavy chain of the AB0045 antibody has the sequence set forth in SEQ ID NO: 49

(MGWSLILLFLVAVATRVHSQVQLQESGPGLVKPSETLSLTCTVSGFSLL
SYGVHWVRQPPGKGLEWLGVIWTGGTTNYNSALMSRFTISKDDSKNTVYL
KMNSLKTEDTAIYYCARYYYGMDYWGQGTLVTVSSASTKGPSVFPLAPCS
RSTSESTAALGCLVKDYFPEPVTVSWNSGALTSGVHTFPAVLQSSGLYSL
SSVVTVPSSSLGTKTYTCNVDHKPSNTKVDKRVESKYGPPCPPCPAPEFL
GGPSVFLFPPKPKDTLMISRTPEVTCWVDVSQEDPEVQFNWYVDGVEVHN
AKTKPREEQFNSTYRVVSVLTVLHQDWLNGKEYKCKVSNKGLPSSIEKTI
SKAKGQPREPQVYTLPPSQEEMTKNQVSLTCLVKGFYPSDIAVEWESNGQ
PENNYKTTPPVLDSDGSFFLYSRLTVDKSRWQEGNVFSCSVMHEALHNHY
TQKSLSLSLGK

(signal sequence underlined; sequence of the constant region presented italics)); the light chain of the AB0045 antibody has the sequence set forth in SEQ ID NO: 50

(MRVPAQLLGLLLLWLPGARCDIQMTQSPSSLSASVGDRVTITCKASQDV
RNTVAWYQQKPGKAPKLLIYSSSYRNTGVPDRFSGSGSGTDFTLTISSLQ
AEDVAVYYCQQHYITPYTFGGGTKVEIKRTVAAPSVFIFPPSDEQLKSGT
ASVVCLLNNFYPREAKVQWKVDNALQSGNSQESVTEQDSKDSTYSLSSTL
TLSKADYEKHKVYACEVTHQGLSSPVTKSFNRGEC

(signal sequence underlined; sequence of the constant region presented italics). Without the signal peptides, the heavy chain of the AB0045 antibody has the sequence set forth in SEQ ID NO: 56

(QVQLQESGPGLVKPSETLSLTCTVSGFSLLSYGVHWVRQPPGKGLEWLG
VIWTGGTTNYNSALMSRFTISKDDSKNTVYLKMNSLKTEDTAIYYCARYY
YGMDYWGQGTLVTVSSASTKGPSVFPLAPCSRSTSESTAALGCLVKDYFP
EPVTVSWNSGALTSGVHTFPAVLQSSGLYSLSSVVTVPSSSLGTKTYTCN
VDHKPSNTKVDKRVESKYGPPCPPCPAPEFLGGPSVFLFPPKPKDTLMIS
RTPEVTCVVVDVSQEDPEVQFNWYVDGVEVHNAKTKPREEQFNSTYRVVS
VLTVLHQDWLNGKEYKCKVSNKGLPSSIEKTISKAKGQPREPQVYTLPPS
QEEMTKNQVSLTCLVKGFYPSDIAVEWESNGQPENNYKTTPPVLDSDGSF
FLYSRLTVDKSRWQEGNVFSCSVMHEALHNHYTQKSLSLSLGK);

and the light chain of the AB0045 antibody has the sequence set forth in SEQ ID NO: 57

(DIQMTQSPSSLSASVGDRVTITCKASQDVRNTVAWYQQKPGKAPKLLIY
SSSYRNTGVPDRFSGSGSGTDFTLTISSLQAEDVAVYYCQQHYITPYTFG
GGTKVEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQ
GLSSPVTKSFNRGEC)

(the constant regions are shown in italics).

The antibodies further include those produced by the hybridoma designated M4, i.e., an antibody containing the heavy chain (IgG2b) sequence:

(SEQ ID NO: 30)
MAVLVLFLCLVAFPSCVLSQVQLKESGPGLVAPSQSLSITCTVSGFSLLS
YGVHWVRQPPGKGLEWLGVIWTGGSTNYNSALMSRLSISKDDSKSQVFLK
MNSLQTDDTAMYYCARYYYAMDYWGQGTSVTVSSAKTTPPSVYPLAPGCG
DTTGSSVTLGCLVKGYFPESVTVTWNSGSLSSSVHTFPALLQSGLYTMSS
SVTVPSSTWPSQTVTCSVAHPASSTTVDKKLEPSGPISTINPCPPCKECH
KCPAPNLEGGPSVFIFPPNIKDVLMISLTPKVTCVVVDVSEDDPDVRISW
FVNNVEVHTAQTQTHREDYNSTIRVVSALPIQHQDWMSGKEFKCKVNNKD
LPSPIERTISKIKGLVRAPQVYILPPPAEQLSRKDVSLTCLVVGFNPGDI
SVEWTSNGHTEENYKDTAPVLDSDGSYFIYSKLDIKTSKWEKTDSFSCNV
RHEGLKNYYLKKTISRSPGK

(signal peptide set forth in underlined text, variable region set forth in plain text, and constant region set forth in italics), and the light chain (kappa) sequence:

MESQIQVFVFVFLWLSGVDGDIVMTQSHKFMFTSVGDRVSITCKASQDVR
NTVAWYQQKTGQSPKLLIYSASYRNTGVPDRFTGSISGTDFTFTISSVQA
EDLALYYCQQHYSTPYTFGGGTKLEVKRADAAPTVSIFPPSSEQLTSGGA
SVVCFLNNFYPKDINVKWKIDGSERQNGVLNSWTDQDSKDSTYSMSSTLT
LTKDEYERHNSYTCEATHKTSTSPIVKSFNRNEC

(signal peptide set forth in underlined text, variable region set forth in plain text, and constant region set forth in italics) (SEQ ID NO: 31). The M4 antibody has a variable heavy chain with an amino acid sequence:

QVQLKESGPGLVAPSQSLSITCTVSGFSLLSYGVHWVRQPPGKGLEWLGV
IWTGGSTNYNSALMSRLSISKDDSKSQVFLKMNSLQTDDTAMYYCARYYY
AMDYWGQGTSVTVSS

(CDRs 1, 2, and 3 (SEQ ID NOs: 34, 35, and 36, respectively) underlined) (SEQ ID NO: 32) and a variable light chain with the amino acid sequence

DIVMTQSHKFMFTSVGDRVSITCKASQDVRNTVAWYQQKTGQSPKLLIYS
ASYRNTGVPDRFTGSISGTDFTFTISSVQAEDLALYYCQQHYSTPYTFGG
GTKLEVK

(CDRs 1, 2, and 3 (SEQ ID NOs: 37, 38, and 39, respectively) underlined) (SEQ ID NO: 33).

The antibodies further include those produced by the hybridoma designated M12, i.e., one with only a kappa chain, having the sequence:

QVFVYMLLWLSGVDGDIVMTQSQKFMSTSVGDRVSVTCKASQNVGTNVAW
YQQKPGQSPKALIYSASYRFSGVPDRFTGSGSGTDFTLTISNVQSEDLAE
YFCQQYNSYPYTFGGGTKLEIKRADAAPTVSIFPPSSEQLTSGGASVVCF
LNNFYPKDINVKWKIDGSERQNGVLNSWTDQDSKDSTYSMSSTLTLTKDE
YERHNSYTCEATHKTSTSPIVKSFNRNEC

(signal peptide set forth in underlined text, variable region set forth in plain text, and constant region set forth in italics) (SEQ ID NO: 40). The M12 antibody has a variable light chain with the amino acid sequence

DIVMTQSQKFMSTSVGDRVSVTCKASQNVGTNVAWYQQKPGQSPKALIYS
ASYRFSGVPDRFTGSGSGTDFTLTISNVQSEDLAEYFCQQYNSYPYTFGG
GTKLEIK

(CDRs 1, 2, and 3 (SEQ ID NOs: 42, 43, and 44, respectively) underlined) (SEQ ID NO: 41).

The antibodies further include the mouse antibody designated AB0046, having a kappa light chain with an amino acid sequence

(SEQ ID NO: 45)
MSSAQFLGLLLLCFQGTRCDIQMTQTTSSLSASLGDRVTISCSASQGISN
YLNWYQQKPDGTFKLLIYYTSILHSGVPSRFSGSGSGTDYSLTISNLEPE
DIATYYCQQYGWLPRTFGGGTKLEIKRADAAPTVSIFPPSSEQLTSGGAS
VVCFLNNFYPKDINVKWKIDGSERQNGVLNSWTDQDSKDSTYSMSSTLTL
TKDEYERHNSYTCEATHKTSTSPIVKSFNRNEC

(signal peptide set forth in underlined text, variable region set forth in plain text, and constant region set forth in italics) and an IgG1 heavy chain with an amino acid sequence

(SEQ ID NO: 46)
MGWSSIILFLVATATGVHSQVQLQQPGSVLVRPGASVKLSCTASGYTFTS
YWMNWVKQRPGQGLEWIGEIYPISGRTNYNEKFKVKATLTVDTSSSTAYM
DLNSLTSEDSAVYYCARSRANWDDYWGQGTTLTVSSAKTTPPSVYPLAPG
SAAQTNSMVTLGCLVKGYFPEPVTVTWNSGSLSSGVHTFPAVLQSDLYTL
SSSVTVPSSTWPSETVTCNVAHPASSTKVDKKIVPRDCGCKPCICTVPEV
SSVFIFPPKPKDVLTITLTPKVTCVVVDISKDDPEVQFSWFVDDVEVHTA
QTQPREEQFNSTFRSVSELPIMHQDWLNGKEFKCRVNSAAFPAPIEKTIS
KTKGRPKAPQVYTIPPPKEQMAKDKVSLTCMITDFFPEDITVEWQWNGQP
AENYKNTQPIMDTDGSYFVYSKLNVQKSNWEAGNTFTCSVLHEGLHNHHT
EKSLSHSPGK

(signal peptide set forth in underlined text, variable region set forth in plain text, and constant region set forth in italics).

The following amino acid sequence comprises the framework regions and complementarity-determining regions (CDRs) of the variable region of the IgG1 heavy chain of AB0046 (with CDRs underlined):

(SEQ ID No: 47)
QVQLQQPGSVLVRPGASVKLSCTASGYTFTSYWMNWVKQRPGQGLEWIG
EIYPISGRTNYNEKFKVKATLTVDTSSSTAYMDLNSLTSEDSAVYYCAR
SRANWDDYWGQGTTLTVSS.

The following amino acid sequence comprises the framework regions and complementarity-determining regions (CDRs) of the variable region of the kappa light chain of AB0046 (with CDRs underlined):

(SEQ ID No: 48)
DIQMTQTTSSLSASLGDRVTISCSASQGISNYLNWYQQKPDGTFKLLIYY
TSILHSGVPSRFSGSGSGTDYSLTISNLEPEDIATYYCQQYGWLPRTFGG
GTKLEIK.

The antibodies for use with the presently provided methods, compositions, and combinations can include any of the antibodies described herein, including antibodies and antibody fragments, including those containing any combination of the various exemplified heavy and light chains, heavy and light chain variable regions, and CDRs. By way of example, the presently provided methods, compositions, and combinations comprise the antibody or antigen binding fragment thereof comprising an amino acid sequence of any of SEQ ID NOs: 1, 2, 3, 4, 5, 6, 7, 8, 9, 10, 11, 12, 13, 14, 15, 16, 17, 18, 30, 31, 32, 33, 34, 35, 36, 37, 38, 39, 40, 41, 42, 43, 44, 45, 46, 47, 48, 49, or 50. Some embodiments provide the methods, compositions, and combinations comprise the antibody or antigen binding fragment thereof comprising the amino acid sequences of SEQ ID NOs: 7 and 12. Certain embodiments provide the methods, compositions, and combinations comprise the antibody or antigen binding fragment thereof comprising the amino acid sequences of SEQ ID NOs: 13, 14, 15, 16, 17, and 18.

Nucleic Acids Encoding Anti-MMP9 Antibodies

The present disclosure provides nucleic acids encoding anti-MMP9 antibodies and functional fragments thereof. Accordingly, the present disclosure provides an isolated polynucleotide (nucleic acid) encoding an antibody or antigen binding fragment as described herein, vectors containing such polynucleotides, and host cells and expression systems for transcribing and translating such polynucleotides into polypeptides.

The present disclosure also contemplates constructs in the form of plasmids, vectors, transcription or expression cassettes which comprise at least one polynucleotide as above.

The present disclosure also provides a recombinant host cell which comprises one or more constructs as above, as well as methods of production of the antibody or antigen binding fragments thereof described herein which method comprises expression of nucleic acid encoding a heavy chain polypeptide and a light chain polypeptide (in the same or different host cells, and from the same or different constructs) in a recombination host cell. Expression can be achieved by culturing under appropriate conditions recombinant host cells containing the nucleic acid. Following production by expression, an antibody or antigen binding fragment can be isolated and/or purified using any suitable technique, then used as appropriate.

Systems for cloning and expression of a polypeptide in a variety of different host cells are well known. Suitable host cells include bacteria, mammalian cells, yeast and baculovirus systems. Mammalian cell lines available in the art for expression of a heterologous polypeptide include Chinese hamster ovary cells, HeLa cells, baby hamster kidney cells, NSO mouse melanoma cells and many others. A common bacterial host is E. coli.

Suitable vectors can be chosen or constructed, containing appropriate regulatory sequences, including operably linked promoter sequences, terminator sequences, polyadenylation sequences, enhancer sequences, marker genes and/or other sequences as appropriate. Vectors can be plasmids, viral e.g. ‘phage, or phagemid, as appropriate. For further details see, for example, Molecular Cloning: a Laboratory Manual: 2nd edition, Sambrook et al., 1989, Cold Spring Harbor Laboratory Press. Many known techniques and protocols for manipulation of nucleic acid, for example in preparation of nucleic acid constructs, mutagenesis, sequencing, introduction of DNA into cells and gene expression, and analysis of proteins, are described in detail in Short Protocols in Molecular Biology, Second Edition, Ausubel et al. eds., John Wiley & Sons, 1992. The disclosures of Sambrook et al. and Ausubel et al. are incorporated herein by reference in their entirety.

The nucleic acid encoding a polypeptide of interest is integrated into the genome of the host cell or can be maintained as a stable or transient episomal element.

Any of a wide variety of expression control sequences—sequences that control the expression of a DNA sequence operatively linked to it—can be used in these vectors to express the DNA sequences. For example, a nucleic acid encoding a polypeptide of interest can be operably linked to a promoter, and provided in an expression construct for use in methods of production of recombinant MMP9 proteins or portions thereof.

Those of skill in the art are aware that nucleic acids encoding the antibody chains disclosed herein can be synthesized using standard knowledge and procedures in molecular biology.

Examples of nucleotide sequences encoding the heavy and light chain amino acid sequences disclosed herein, are as follows:

VH1:

(SEQ ID NO: 19)
CAGGTGCAGC TGCAGGAATC CGGCCCTGGC CTGGTCAAGC
CCTCCGAGAC ACTGTCCCTG ACCTGCACCG TGTCCGGCTT
CTCCCTGCTG TCCTACGGCG TGCACTGGGT CCGACAGCCT
CCAGGGAAGG GCCTGGAATG GCTGGGCGTG ATCTGGACCG
GCGGCACCAC CAACTACAAC TCCGCCCTGA TGTCCCGGCT
GACCATCTCC AAGGACGACT CCAAGTCCAC CGTGTACCTG
AAGATGAACT CCCTGAAAAC CGAGGACACC GCCATCTACT
ACTGCGCCCG GTACTACTAC GGCATGGACT ACTGGGGCCA
GGGCACCTCC GTGACCGTGT CCTCA

VH2:

(SEQ ID NO: 20)
CAGGTGCAGC TGCAGGAATC CGGCCCTGGC CTGGTCAAGC
CCTCCGAGAC ACTGTCCCTG ACCTGCACCG TGTCCGGCTT
CTCCCTGCTG TCCTACGGCG TGCACTGGGT CCGACAGCCT
CCAGGCAAAG GCCTGGAATG GCTGGGCGTG ATCTGGACCG
GCGGCACCAC CAACTACAAC TCCGCCCTGA TGTCCCGGCT
GACCATCTCC AAGGACGACT CCAAGAACAC CGTGTACCTG
AAGATGAACT CCCTGAAAAC CGAGGACACC GCCATCTACT
ACTGCGCCCG GTACTACTAC GGCATGGACT ACTGGGGCCA
GGGCACCCTG GTCACCGTGT CCTCA

VH3:

(SEQ ID NO: 21)
CAGGTGCAGC TGCAGGAATC CGGCCCTGGC CTGGTCAAGC
CCTCCGAGAC ACTGTCCCTG ACCTGCACCG TGTCCGGCTT
CTCCCTGCTG TCCTACGGCG TGCACTGGGT CCGACAGCCT
CCAGGCAAAG GCCTGGAATG GCTGGGCGTG ATCTGGACCG
GCGGCACCAC CAACTACAAC TCCGCCCTGA TGTCCCGGTT
CACCATCTCC AAGGACGACT CCAAGAACAC CGTGTACCTG
AAGATGAACT CCCTGAAAAC CGAGGACACC GCCATCTACT
ACTGCGCCCG GTACTACTAC GGCATGGACT ACTGGGGCCA
GGGCACCCTG GTCACCGTGT CCTCA

VH4:

(SEQ ID NO: 22)
CAGGTGCAGC TGCAGGAATC CGGCCCTGGC CTGGTCAAGC
CCTCCGAGAC ACTGTCCCTG ACCTGCACCG TGTCCGGCTT
CTCCCTGCTG TCCTACGGCG TGCACTGGGT CCGACAGCCT
CCAGGCAAAG GCCTGGAATG GCTGGGCGTG ATCTGGACCG
GCGGCACCAC CAACTACAAC TCCGCCCTGA TGTCCCGGTT
CACCATCTCC AAGGACGACT CCAAGAACAC CCTGTACCTG
AAGATGAACT CCCTGAAAAC CGAGGACACC GCCATCTACT
ACTGCGCCCG GTACTACTAC GGCATGGACT ACTGGGGCCA
GGGCACCCTG GTCACCGTGT CCTCA

Vk1:

(SEQ ID NO: 23)
GACATCGTGA TGACCCAGTC CCCCAGCTTC CTGTCCGCCT
CCGTGGGCGA CAGAGTGACC ATCACATGCA AGGCCTCTCA
GGACGTGCGG AACACCGTGG CCTGGTATCA GCAGAAAACC
GGCAAGGCCC CCAAGCTGCT GATCTACTCC TCCTCCTACC
GGAACACCGG CGTGCCCGAC CGGTTTACCG GCTCTGGCTC
CGGCACCGAC TTTACCCTGA CCATCAGCTC CCTGCAGGCC
GAGGACGTGG CCGTGTACTT CTGCCAGCAG CACTACATCA
CCCCCTACAC CTTCGGCGGA GGCACCAAGG TGGAAATAAA A

Vk2:

(SEQ ID NO: 24)
GACATCGTGA TGACCCAGTC CCCCTCCAGC CTGTCCGCCT
CTGTGGGCGA CAGAGTGACC ATCACATGCA AGGCCTCTCA
GGACGTGCGG AACACCGTGG CCTGGTATCA GCAGAAGCCC
GGCAAGGCCC CCAAGCTGCT GATCTACTCC TCCTCCTACC
GGAACACCGG CGTGCCCGAC CGGTTTACCG GCTCTGGCTC
CGGCACCGAC TTTACCCTGA CCATCAGCTC CCTGCAGGCC
GAGGACGTGG CCGTGTACTT CTGCCAGCAG CACTACATCA
CCCCCTACAC CTTCGGCGGA GGCACCAAGG TGGAAATAAA A

Vk3:

(SEQ ID NO: 25)
GACATCCAGA TGACCCAGTC CCCCTCCAGC CTGTCCGCCT
CTGTGGGCGA CAGAGTGACC ATCACATGCA AGGCCTCCCA
GGACGTGCGG AACACCGTGG CCTGGTATCA GCAGAAGCCC
GGCAAGGCCC CCAAGCTGCT GATCTACTCC TCCTCCTACC
GGAACACCGG CGTGCCCGAC CGGTTCTCTG GCTCTGGAAG
CGGCACCGAC TTTACCCTGA CCATCAGCTC CCTGCAGGCC
GAGGACGTGG CCGTGTACTT CTGCCAGCAG CACTACATCA
CCCCCTACAC CTTCGGCGGA GGCACCAAGG TGGAAATAAA A

Vk4:

(SEQ ID NO: 26)
GACATCCAGA TGACCCAGTC CCCCTCCAGC CTGTCCGCCT
CTGTGGGCGA CAGAGTGACC ATCACATGCA AGGCCTCTCA
GGACGTGCGG AACACCGTGG CCTGGTATCA GCAGAAGCCC
GGCAAGGCCC CCAAGCTGCT GATCTACTCC TCCTCCTACC
GGAACACCGG CGTGCCCGAC CGGTTCTCTG GCTCTGGAAG
CGGCACCGAC TTTACCCTGA CCATCAGCTC CCTGCAGGCC
GAGGACGTGG CCGTGTACTA CTGCCAGCAG CACTACATCA
CCCCCTACAC CTTCGGCGGA GGCACCAAGG TGGAAATAAA A

Because the structure of antibodies, including the juxtaposition of CDRs and framework regions in the variable region, the structure of framework regions and the structure of heavy- and light-chain constant regions, is well-known in the art; it is well within the skill of the art to obtain related nucleic acids that encode anti-MMP-9 antibodies. Accordingly, polynucleotides comprising nucleic acid sequences having at least 75%, at least 80%, at least 85%, at least 90%, at least 95%, at least 98% and at least 99% homology to any of the nucleotide sequences disclosed herein are also provided. Accordingly, polynucleotides comprising nucleic acid sequences having at least 75%, at least 80%, at least 85%, at least 90%, at least 95%, at least 98% and at least 99% identity to any of the nucleotide sequences disclosed herein are also provided. In one example, the polynucleotide contains at least at or about 75%, 80%, 85%, 90%, 91%, 92%, 93%, 94%, 95%, 96%, 97%, 98%, 99% or more sequence identity with SEQ ID NO: 21 or includes or is SEQ ID NO: 21 and/or contains at least at or about 75%, 80%, 85%, 90%, 91%, 92%, 93%, 94%, 95%, 96%, 97%, 98%, 99% or more sequence identity with SEQ ID NO: 26 or includes or is SEQ ID NO: 26.

### Pharmaceutical Compositions

MMP9 binding proteins, as well as nucleic acid (e.g., DNA or RNA) encoding MMP9 binding proteins, can be provided as a pharmaceutical composition, e.g., combined with a pharmaceutically acceptable carrier or excipient. Such pharmaceutical compositions are useful for, for example, administration to a subject in vivo or ex vivo, and for diagnosing and/or treating a subject with the MMP9 binding proteins, such as in any of the therapeutic or diagnostic methods provided herein.

Pharmaceutically acceptable carriers or excipients are physiologically acceptable to the administered patient and retain the therapeutic properties of the antibodies or peptides with which it is administered. Pharmaceutically-acceptable carriers or excipients and their formulations are and generally described in, for example, Remington' pharmaceutical Sciences (18th Edition, ed. A. Gennaro, Mack Publishing Co., Easton, Pa. 1990). One exemplary pharmaceutical carrier is physiological saline. Each carrier or excipient is “pharmaceutically acceptable” in the sense of being compatible with the other ingredients of the formulation and not substantially injurious to the patient.

Pharmaceutical compositions can be formulated to be compatible with a particular route of administration, systemic or local. Thus, pharmaceutical compositions include carriers, diluents, or excipients suitable for administration by various routes.

Pharmaceutical compositions can include pharmaceutically acceptable additives. Examples of additives include, but are not limited to, a sugar such as mannitol, sorbitol, glucose, xylitol, trehalose, sorbose, sucrose, galactose, dextran, dextrose, fructose, lactose and mixtures thereof. Pharmaceutically acceptable additives can be combined with pharmaceutically acceptable carriers and/or excipients such as dextrose. Additives also include surfactants such as polysorbate 20 or polysorbate 80.

The formulation and delivery methods will generally be adapted according to the site and the disease to be treated. Exemplary formulations include, but are not limited to, those suitable for parenteral administration, e.g., intravenous, intra-arterial, intramuscular, or subcutaneous administration, or oral administration. In one embodiment, the anti-MMP9 antibody or antigen binding fragment thereof, the composition or the formulation thereof is delivered by intravenous administration (which may be referred to as intravenous infusion). In some embodiment, the anti-MMP9 antibody or antigen binding fragment thereof, the composition or the formulation thereof is delivered by subcutaneous administration (which may be referred to as subcutaneous injection).

Pharmaceutical compositions for parenteral delivery include, for example, water, saline, phosphate buffered saline, Hank's solution, Ringer's solution, dextrose/saline, and glucose solutions. The formulations can contain auxiliary substances to approximate physiological conditions, such as buffering agents, tonicity adjusting agents, wetting agents, detergents and the like. Additives can also include additional active ingredients such as bactericidal agents, or stabilizers. For example, the solution can contain sodium acetate, sodium lactate, sodium chloride, potassium chloride, calcium chloride, sorbitan monolaurate or triethanolamine oleate. Additional parenteral formulations and methods are described in Bai (1997) J. Neuroimmunol. 80:65 75; Warren (1997) J. Neurol. Sci. 152:31 38; and Tonegawa (1997) J. Exp. Med. 186:507 515. The parenteral preparation can be enclosed in ampules, disposable syringes or multiple dose vials made of glass or plastic.

Pharmaceutical compositions for intravenous, intradermal or subcutaneous administration can include a sterile diluent, such as water, saline solution, fixed oils, polyethylene glycols, glycerine, propylene glycol or other synthetic solvents; antibacterial agents such as benzyl alcohol or methyl parabens; antioxidants such as ascorbic acid, glutathione or sodium bisulfite; chelating agents such as ethylenediaminetetraacetic acid; buffers such as acetates, citrates or phosphates and agents for the adjustment of tonicity such as sodium chloride or dextrose.

Pharmaceutical compositions for injection include aqueous solutions (where water soluble) or dispersions and sterile powders for the extemporaneous preparation of sterile injectable solutions or dispersion. For intravenous administration, suitable carriers include physiological saline, bacteriostatic water, Cremophor ELTM (BASF, Parsippany, N.J.) or phosphate buffered saline (PBS). The carrier can be a solvent or dispersion medium containing, for example, water, ethanol, polyol (for example, glycerol, propylene glycol, and liquid polyetheylene glycol, and the like), and suitable mixtures thereof. Fluidity can be maintained, for example, by the use of a coating such as lecithin, by the maintenance of the required particle size in the case of dispersion and by the use of surfactants. Antibacterial and antifungal agents include, for example, parabens, chlorobutanol, phenol, ascorbic acid and thimerosal. Isotonic agents, for example, sugars, polyalcohols such as mannitol, sorbitol, and sodium chloride may be included in the composition. The resulting solutions can be packaged for use as is, or lyophilized; the lyophilized preparation can later be combined with a sterile solution prior to administration.

Pharmaceutically acceptable carriers can contain a compound that stabilizes, increases or delays absorption or clearance. Such compounds include, for example, carbohydrates, such as glucose, sucrose, or dextrans; low molecular weight proteins; compositions that reduce the clearance or hydrolysis of peptides; or excipients or other stabilizers and/or buffers. Agents that delay absorption include, for example, aluminum monostearate and gelatin. Detergents can also be used to stabilize or to increase or decrease the absorption of the pharmaceutical composition, including liposomal carriers. To protect from digestion the compound can be complexed with a composition to render it resistant to acidic and enzymatic hydrolysis, or the compound can be complexed in an appropriately resistant carrier such as a liposome. Means of protecting compounds from digestion are known in the art (see, e.g., Fix (1996) Pharm Res. 13:1760 1764; Samanen (1996) J. Pharm. Pharmacol. 48:119 135; and U.S. Pat. No. 5,391,377, describing lipid compositions for oral delivery of therapeutic agents).

Compositions of the present invention can be combined with other therapeutic moieties or imaging/diagnostic moieties as provided herein. Therapeutic moieties and/or imaging moieties can be provided as a separate composition, or as a conjugated moiety present on an MMP9 binding protein.

Formulations for in vivo administration are generally sterile. In one embodiment, the pharmaceutical compositions are formulated to be free of pyrogens such that they are acceptable for administration to human patients.

Various other pharmaceutical compositions and techniques for their preparation and use will be known to those of skill in the art in light of the present disclosure. For a detailed listing of suitable pharmacological compositions and associated administrative techniques one can refer to the detailed teachings herein, which can be further supplemented by texts such as Remington: The Science and Practice of Pharmacy 20th Ed. (Lippincott, Williams & Wilkins 2003).

Pharmaceutical compositions can be formulated based on the physical characteristics of the patient/subject needing treatment, the route of administration, and the like. Such can be packaged in a suitable pharmaceutical package with appropriate labels for the distribution to hospitals and clinics wherein the label is for the indication of treating a disorder as described herein in a subject. Medicaments can be packaged as a single or multiple units. Instructions for the dosage and administration of the pharmaceutical compositions of the present invention can be included with the pharmaceutical packages and kits described below.

### Methods of Use

The MMP9 binding proteins, including anti-MMP9 antibodies and fragments thereof, of the present disclosure can be used, for example, in therapeutic and diagnostic methods, such as methods of detection of MMP9 in a sample, methods of treatment (e.g., as in methods of inhibition of angiogenesis), and methods of diagnosis and prognosis. Thus, provided are diagnostic and therapeutic methods and uses of the anti-MMP9 antibodies. Examples of methods of use are described below.

Methods of Treatment

Provided herein are methods of treatment and/or prevention of chronic obstructive pulmonary disease (COPD).

As used herein, the term “treatment” refers to clinical intervention (such as, e.g., administration of an MMP9 binding protein described herein) designed to alter the natural course of the individual or cell being treated during the course of clinical pathology of COPD. Desirable effects of treatment include decreasing the rate of disease progression or mortality, ameliorating or palliating the disease state, and remission or improved prognosis. In some embodiments, the treatment improves COPD symptoms, reduces frequency or severity of COPD exacerbations, improves lung function, patient-reported COPD symptoms (such as exercise tolerance and quality of life). A response is achieved when the patient experiences partial or total alleviation, or reduction of signs or symptoms of illness, and specifically includes, without limitation, prolongation of survival and improved quality of life. A subject is successfully “treated,” for example, if one or more symptoms associated with COPD are mitigated or eliminated.

As used herein, “in conjunction with” refers to administration of one treatment modality in addition to another treatment modality. As such, “in conjunction with” refers to administration of one treatment modality (e.g., an MMP9 binding protein described herein) before, during or after administration of the other treatment modality to the individual.

As used herein, the term “prevention” includes providing prophylaxis with respect to occurrence or recurrence of COPD in an individual. An individual may be predisposed to, susceptible to COPD, or at risk of developing COPD, but has not yet been diagnosed with the disorder. In some embodiments, MMP9 binding proteins described herein are used to delay development of COPD. In some embodiments, the MMP9 binding proteins described herein prevents COPD exacerbations and/or decline in lung function or lung tissue breakdown.

As used herein, an individual “at risk” of developing COPD may or may not have detectable disease or symptoms of disease, and may or may not have displayed detectable disease or symptoms of disease prior to the treatment methods described herein. “At risk” denotes that an individual has one or more risk factors, which are measurable parameters that correlate with development of COPD, as known in the art. An individual having one or more of these risk factors has a higher probability of developing COPD than an individual without one or more of these risk factors.

An “effective amount” refers to at least an amount effective, at dosages and for periods of time necessary, to achieve the desired or indicated effect, including a therapeutic or prophylactic result. An effective amount can be provided in one or more administrations.

A “therapeutically effective amount” is at least the minimum concentration required to effect a measurable improvement of a particular disorder. A therapeutically effective amount herein may vary according to factors such as the disease state, age, sex, and weight of the patient, and the ability of the antibody to elicit a desired response in the individual. A therapeutically effective amount is also one in which any toxic or detrimental effects of the antibody are outweighed by the therapeutically beneficial effects.

Thus, an effective amount of an agent, when administered (either alone or in combination with another therapeutic agent, as may be specified) to a subject is effective to prevent or ameliorate the disease condition or the progression of the disease, or result in amelioration of symptoms, e.g., treatment, healing, prevention or amelioration of the relevant medical condition, or an increase in rate of treatment, healing, prevention or amelioration of such conditions. When applied to an individual active ingredient administered alone, a therapeutically effective dose refers to that ingredient alone. When applied to a combination, a therapeutically effective dose refers to combined amounts of the active ingredients that result in the therapeutic effect, whether administered in combination, serially or simultaneously.

A “Prophylactically effective amount” refers to an amount effective, at the dosages and for periods of time necessary, to achieve the desired prophylactic result. Typically but not necessarily, since a prophylactic dose is used in subjects prior to or at the earlier stage of disease, the prophylactically effective amount can be less than the therapeutically effective amount.

In general, MMP9 binding proteins are administered in a therapeutically effective amount, e.g., in an amount to effect inhibition of MMP9 activity, or to treat COPD.

In certain embodiments, an MMP9 binding protein described herein (e.g., an antibody that binds MMP9 or a functional fragment thereof) is administered at the interval of one, two or three weeks, or once every one, two, or three weeks. In certain embodiments, an MMP9 binding protein described herein (e.g., an antibody that binds MMP9 or a functional fragment thereof) can be administered daily or less frequently than daily, for example, six times a week, five times a week, four times a week, three times a week, twice a week, once a week, once every two weeks, once every three weeks, once a month, once every two months, once every three months, or once every six months. In some embodiments, the treatment includes at least one, at least two, at least three, at least four, at least five, at least six, at least seven, at least eight, at least nine, or at least ten administration. The compositions may also be administered in a sustained release formulation, such as in an implant which gradually releases the composition for use over a period of time, and which allows for the composition to be administered less frequently, such as once a month, once every 2-6 months, once every year, or even a single administration. Also, the treatment is continuous. In one embodiment, the anti-MMP9 antibody or antigen-binding fragment thereof, the composition or the formulation thereof is administered once a week. In certain embodiment, the anti-MMP9 antibody or antigen-binding fragment thereof, the composition or the formulation thereof is administered once every two weeks.

The MMP9 binding proteins can be administered to an individual via any route, including, but not limited to, intravenous (e.g., by infusion pumps), intraperitoneal, intra-arterial, intrapulmonary, oral, inhalation, intravesicular, intramuscular, intra-tracheal, subcutaneous, intrathecal, transdermal, transpleural, topical, inhalational (e.g., as mists of sprays), mucosal (such as via nasal mucosa), subcutaneous, transdermal, gastrointestinal, intraarticular, intracisternal, or intraventricular. In some embodiments, the compositions are administered systemically (for example by intravenous injection). In some embodiments, the MMP9 binding proteins are administered locally (for example by intra-arterial or injection). In some embodiments, the MMP9 binding proteins are administered subcutaneously. In some embodiments, the MMP9 binding proteins are administered intradermally. In some embodiments, the MMP9 binding proteins are administered via inhalation. In some embodiments, the MMP9 binding proteins are administered mucosally. In one embodiment, the anti-MMP9 antibody or antigen-binding fragment thereof, the composition or the formulation thereof is delivered by intravenous administration (i.e. intravenous infusion) twice every two weeks. In certain embodiment, the anti-MMP9 antibody or antigen-binding fragment thereof, the composition or the formulation thereof is delivered by subcutaneous administration once every week.

In some embodiments, the antibody that binds MMP9 or antigen binding fragment thereof is administered at about 25 mg per subject to about 800 mg per subject. In some embodiments, the antibody that binds MMP9 or antigen binding fragment thereof is administered at about 50 mg, about 100 mg, about 200 mg, about 300 mg, about 400 mg, about 500 mg, about 600 mg, about 700 mg, or about 800 mg per subject, including any range in between these values. In certain embodiments, the antibody that binds MMP9 (i.e. anti-MMP9 antibody) or antigen binding fragment thereof is administered at about 150 mg, about 250 mg, about 350 mg, about 450 mg, about 550 mg, about 650 mg, or about 750 mg per subject, including any range in between these values. In some embodiments, the antibody or the antigen binding fragment thereof of the above dosage is administered once a week, once every two weeks, once every three weeks, once a month, once every two months, once every three months, or once every six months. In some embodiments, the antibody that binds MMP9 or antigen binding fragment thereof is administered at about 400 mg every two weeks. In certain embodiments, the antibody that binds to MMP9 or the antigen binding fragment thereof is administered to the subject at a dosage of about 200 mg every two weeks. In certain embodiment, the antibody that binds MMP9 or antigen binding fragment thereof is administered at about 150 mg once a week. In certain embodiment, the antibody that binds MMP9 or antigen binding fragment thereof is administered at about 300 mg once a week. In certain embodiments, the antibody that binds to MMP9 or the antigen binding fragment thereof is administered to the subject in a two-step procedure: first, a loading dose phase (more frequent dosing to cover the “target sink”/“tissue and serum sink” or high baseline concentration of MMP9 associated with the disease, wherein the dosing range is administered to the subject at a dosage of about 200 mg, about 300 mg, or about 400 mg every week for an interval of one, two or three weeks, or more frequent dosing to cover the “target sink” or high baseline concentration of MMP9 associated with the disease) and second, once a predictable pK has been established after the loading dose phase, a lower weekly dose such as 150, 125, 100 or 50 mg/week. In some embodiments, the lower weekly dose could be lower on a weekly basis, e.g., 150, 125, 100 or 50 mg/week. In one embodiment, the anti-MMP9 antibody or antigen-binding fragment thereof, the composition or the formulation thereof is administered intravenously (i.e. intravenous infusion) at about 400 mg every two weeks. In one embodiment, the anti-MMP9 antibody or antigen-binding fragment thereof, the composition or the formulation thereof is administered intravenously at about 200 mg every two weeks. In one embodiment, the anti-MMP9 antibody or antigen-binding fragment thereof, the composition or the formulation thereof is administered subcutaneously (i.e. subcutaneous injection) at about 150 mg once a week. In one embodiment, the anti-MMP9 antibody or antigen-binding fragment thereof, the composition or the formulation thereof is administered subcutaneously at about 300 mg every two weeks.

The selected dosage regimen will depend upon a variety of factors including the activity of the MMP9 binding protein, the route of administration, the time of administration, the rate of excretion of the particular compound being employed, the duration of the treatment, other drugs, compounds and/or materials used in combination with the particular composition employed, the age, sex, weight, condition, general health and prior medical history of the patient being treated, and like factors well known in the medical arts.

In some embodiments, dosage is determined based on a pharmacokinetic model for antibodies displaying target-mediated disposition. In contrast to the relatively linear pharmacokinetics observed for antibodies directed to soluble receptor targets, antibodies directed toward tissue-based target receptors frequently demonstrate non-linear pharmacokinetics. Mager, D. E. (2006), Adv Drug Deliv Rev 58(12-13): 1326-1356. The basis for non-linear disposition relates to the high affinity binding of antibody to target and the extent of binding (relative to dose), such that the interaction is reflected in the pharmacokinetic characteristics of the antibody. Mager, D. E. and W. J. Jusko (2001), J Pharmacokinet Pharmacodyn 28(6): 507-532. Included within target mediated drug disposition is receptor-mediated endocytosis (internalization) of the antibody-receptor complex. Wang, W., E. Q. Wang, et al. (2008), Clin Pharmacol Ther 84(5): 548-558.

In a pharmacokinetic model for an antibody having target-mediated disposition, in the absence of drug (antibody), the target receptor is synthesized at a constant rate and eliminated by a first-order process. As a result, the target receptor exists at a steady-state concentration in the absence of drug (antibody). When drug is added to the body it can interact with the target receptor in a bimolecular reaction, distribute into less well perfused tissue, or be eliminated via first-order processes. At low drug concentrations the predominant movement of drug is onto the receptor due to the high affinity binding. As the amount of drug entering the body becomes sufficient to bind the available mass of receptor the drug distributes into and out of tissue and is eliminated. As drug concentrations fall and drug equilibrates from tissue this provides an additional reservoir to binding newly synthesized receptor.

A clinician having ordinary skill in the art can readily determine and prescribe the effective amount (ED50) of the pharmaceutical composition required. For example, the physician or veterinarian can start doses of the compounds of the invention employed in the pharmaceutical composition at levels lower than that required in order to achieve the desired therapeutic effect and gradually increase the dosage until the desired effect is achieved.

In some cases, the methods of treatment include parenteral administration, e.g., intravenous, intra-arterial, intradermal, intramuscular, or subcutaneous administration, or oral administration of the agent, e.g., anti-MMP9 antibody or composition containing the same.

As used herein, the term “subject” means a mammalian subject. Exemplary subjects include, but are not limited to humans, monkeys, dogs, cats, mice, rats, cows, horses, goats and sheep. In some embodiments, the subject has COPD, and can be treated with the agent of the present invention as described below. In certain embodiment, the subject is a patient being diagnosed or suspected of having COPD. If needed, for treatments, methods can further include additional therapies, such as those listed below. Administration of other anti-COPD agents or treatments can be concurrent or sequential with administration of the compositions disclosed herein. Also, the subject having COPD may have received or may not have received any prior COPD treatment.

In some embodiments, the antibody that binds MMP9 or antigen binding fragment thereof is administered alone, as a monotherapy. In other embodiments, the antibody is administered as part of a combination therapy with one or more other therapeutic agents for treating COPD. The therapeutic agents include but are not limited to 1) Short-acting β2 agonists (such as, for example, salbutamol (albuterol), levalbuterol, fenoterol, terbutaline), 2) short-acting anticholinergics (such as, for example, ipratropium bromide, oxitropium bromide), 3) Long-acting β-2 agonists (such as, for example, formoterol, arfomoterol, indacaterol, salmeterol, tulobuterol), 4) Long-acting anticholinergics (such as, for example, aclidinium bromide, glycopyrronium bromide, tiotropium), 5) Combination short-acting β-2 agonist plus anticholinergics (such as, for example, fenoterol/ipratropium, salbutamol/ipratropium), 6) Inhaled corticosteroids (such as, for example, beclomethasone, budesonide, fluticasone), 7) Combination long-acting β-2 agonists plus corticosteroids (such as, for example, formoterol/budesonide, formoterol/mometasone, salmeterol/fluticasone), 8) Methylxanthines (such as, for example, aminophylline, theophylline), 9) Phosphosdiesterase-4 inhibitors (such as roflumilast), 10) Systemic corticosteroids (such as, for example, prednisone, methylprednisolone). In the combination therapy, the antibody of the present application may be used as the primary or front-line agent or the secondary or additional agent in treating patients in need thereof. In some embodiments, the antibody of the present application may be used as the secondary or additional agent in treating patients in need thereof. In one embodiment, the MMP9 antibody or antigen binding fragment thereof may be combined with the therapeutic agents selected from the group consisting of short-acting β-2 agonists, short-acting anticholinergics, long-acting β-2 agonists, and long-acting anticholinergics; and any of the combinations of anti-MMP9 antibody or antigen binding fragment thereof and the one or more therapeutic agents may be further combined with β-2 agonist, anticholinergics, inhaled corticosteroids, systemic corticosteroids, methylxanthines, or phosphosdiesterase-4 inhibitors. By way of example, the MMP9 antibody or antigen binding fragment thereof may be combined with β-2 agonist (either long or short acting) and methylxanthines.

In some aspects, the dosage of antibody that binds MMP9 or the antigen binding fragment thereof can be adjusted and administered at about 133, about 267, about 400, about 600 or about 1200 mg/Kg body weight, including any range in between these values. After each therapeutic cycle, the patients are monitored for the levels of MMP9 antibodies, MMP9, or other suitable biomarkers.

The agents in a combination therapy can be administered, via a suitable route described above, simultaneously (in the same composition or separately), or sequentially, in any order.

In some embodiments, the treatment methods include steps for monitoring treatment, including for monitoring efficacy or activity, such as pharmacodynamic activity. In some examples, such methods include detecting or measuring the presence, absence, levels, and/or expression of markers, such as cytokines and other inflammatory markers that are indicative of efficacy of treatment, and in addition can act as substrates of MMP9 released into serum directly via cleavage or from stores in the extracellular matrix by MMP9 activity, in biological test samples obtained from subjects being treated using the methods and compositions. The samples typically are blood samples or serum samples but can include other biological samples as described herein. In one embodiment, the biological test sample is sputum obtained from subject being diagnosed or suspected to have COPD using the methods of the present application. Among the markers for use in such methods are Tissue Inhibitor of Metalloproteinases 1 (TIMP-1), Tumor Necrosis Factor alpha (TNF-alpha), Macrophage Inflammatory Protein-2 (MIP-2), Interleukin-17A (IL-17A), CXCL10, Lymphotactin, Macrophage Inflammatory Protein-1 beta (MIP-1 beta), Oncostatin-M (OSM), Interleukin-6 (IL-6), Interleukin-8 (IL-8), Monocyte Chemotactic Protein 3 (MCP-3), Vascular Endothelial Growth Factor A (VEGF-A), Monocyte Chemotactic Protein-5 (MCP-5), Interleukin-1 alpha (IL-1 alpha), Macrophage Colony-Stimulating Factor-1 (M-CSF-1), Myeloperoxidase (MPO), Growth-Regulated Alpha Protein (KC/GRO), Interleukin-7 (IL-7), Leukemia Inhibitory Factor (LIF), Apolipoprotein A-I (Apo A-I), C-Reactive Protein (CRP), Granulocyte Chemotactic Protein-2 (GCP-2), Interleukin-11 (IL-11), Monocyte Chemotactic Protein 1 (MCP-1), von Willebrand factor (vWF), RANK ligand, CCL-5, and Stem Cell Factor (SCF) gene products. Other markers used in such methods may include desmosine, elastin and collagen fragments generated by MMP9 activity. These markers are reflective of local tissue destruction caused by the disease process. Such products of local tissue destruction may end up in and may be detectable in serum.

In some embodiments, after each therapeutic cycle, the patients are monitored for the levels of MMP9 antibodies, MMP9, or other suitable biomarkers.

Among the provided methods are those that provide improved safety profiles compared to available treatments and therapeutic regimens and/or sustained long-term efficacy in treating COPD.

Chronic Obstructive Pulmonary Disorder (COPD)

MMP9 binding proteins described herein, such as antibodies that bind MMP9 and fragments thereof, are used in the treatment or prevention of chronic obstructive pulmonary disorder (COPD), e.g., by inhibiting MMP9 in subjects having COPD.

COPD, also known as chronic obstructive lung disease (COLD) or chronic obstructive airway disease (COAD), refers to a group of progressive obstructive lung diseases (e.g. emphysema and chronic bronchitis) that are characterized by poor airflow (airflow limitation) and inability to breathe out fully (air trapping). The poor airflow is the result of destruction of the connective tissue of the lungs, which then contributes to the poor airflow and poor absorption and release of respiratory gases. Signs and symptoms of COPD include, but are not limited to, e.g., shortness of breath, especially during physical activities, wheezing, chest tightness, excess mucus in the lungs, a chronic cough that produces sputum that may be clear, white, yellow or greenish, cyanosis (i.e., blueness of the lips and fingernail beds), frequent respiratory infections, fatigue, and, in the later stages, unintended weight loss. Subjects having COPD can experience episodes called exacerbations, during which their symptoms become worse and persist for days or longer. Subjects who experience frequent exacerbations have a faster deterioration of their lung function.

COPD develops as a significant and chronic inflammatory response to inhaled irritants. Known causes of COPD include tobacco smoking, air pollution (e.g., exposure to poorly ventilated cooking fires, often fueled by coal or biomass fuels such as wood or animal dung, urban air pollution, exhaust gas), and occupational exposures (e.g., such as prolonged exposure to workplace dusts, chemicals, and fumes). Genetics may play a role in the development of COPD. Currently, the only inherited risk factor is alpha-1 antitrypsin (AAT) deficiency. Exacerbation (i.e., a sudden worsening of symptoms) is typically triggered by infection, environmental pollutants, or improper use of medications.

COPD is typically diagnosed using spirometry (i.e., pulmonary function tests that measure lung function, specifically the amount and or speed of air that can be inhaled and exhaled). The spirometric components that are measured to make the diagnosis are: a) the forced expiratory volume in one second (FEV₁), which is the greatest volume of air that can be breathed out in the first second of a breath; b) the forced vital capacity (FVC), which is the greatest volume of air that can be breathed out in a single large breath. An FEV₁/FVC ratio of less than 70% in someone with symptoms of COPD defines a person as having the disease. In the elderly, diagnostic criteria additionally require a FEV₁ of less than 80% of predicted.

Additional tests are used to assess the severity of COPD in a subject. For example, the modified British Medical Research Council questionnaire (mMRC) or the COPD assessment test (CAT) are simple questionnaires that may be used to determine the severity of symptoms. Scores on CAT range from 0-40 with the higher the score, the more severe the disease. Spirometry may help to determine the severity of airflow limitation. This is typically based on the FEV₁ expressed as a percentage of the predicted “normal” for the person's age, gender, height and weight. Weight loss and muscle weakness, as well as the presence of other diseases, should also be taken into account. Additional criteria for assessing the severity of COPD in a subject are described in Vestbo (2013) “Diagnosis and Assessment.” Global Strategy for the Diagnosis, Management, and Prevention of Chronic Obstructive Pulmonary Disease. Global Initiative for Chronic Obstructive Lung Disease. pp. 9-17.

Chest X-rays and/or chest CT scans and an arterial blood gas test are generally performed to exclude other conditions at the time of diagnosis. Characteristic signs on X-ray are overexpanded lungs, a flattened diaphragm, increased retrosternal airspace, and bullae (fluid-filled sacs in the lungs). Analysis of arterial blood gas is used to determine the need for oxygen and is typically performed in subjects with an FEV₁ less than 35% predicted, subjects with a peripheral oxygen saturation of less than 92% and subjects with symptoms of congestive heart failure. In areas of the world were alpha-1 antitrypsin deficiency is common, people with COPD (particularly those below the age of 45 and with emphysema affecting the lower parts of the lungs) are considered for testing.

In some embodiments the subject to be treated with an MMP9 binding protein described herein has any one or more of the following symptoms: shortness of breath, especially during physical activities, wheezing, chest tightness, excess mucus in the lungs, a chronic cough that produces sputum that may be clear, white, yellow or greenish, cyanosis (i.e., blueness of the lips and fingernail beds), frequent respiratory infections, fatigue, and unintended weight loss. In some embodiments, the subject to be treated has COPD exacerbations. In some embodiments, the subject has an FEV₁≧40%© and less than 80% predicted. In some embodiments, the subject has an FEV₁/FVC ratio of less than 70%. In some embodiments, the subject has been diagnosed with COPD based on results of the modified British Medical Research Council questionnaire (mMRC). In some embodiments, the subject has been diagnosed with COPD based on the COPD assessment test (CAT). In some embodiments, the subject has been diagnosed with COPD based on Global Initiative for Chronic Obstructive Lung Disease (GOLD) guidelines. In some embodiments, the subject has been diagnosed with COPD based on one or more of the following: chest X-ray, chest CT scan, and analysis arterial blood gas. In one embodiment, the subject is diagnosed with COPD having two or more exacerbations in a year prior to the treatments of the present application. In some embodiments, the subject is diagnosed with COPD having two or more exacerbations in a year prior to the treatments and reduced lung function. The reduced lung function may be determined by the clinical indications described herein. In some embodiment, the reduced lung function may be determined by an FEV₁ less than 80%. The subject diagnosed with COPD having two or more exacerbation in a year account for majority of morbidity, mortality, as well as costs associated with exacerbation-related hospitalizations. Without being bound to any hypothesis, such patients may have increased MMP9 levels or activities in the airway compared to those of healthy subject or the COPD subjects having one or less exacerbation. The MMP9 levels or activities in the airway may be determined by a person skilled in the art using commonly-used methods. In some embodiments, the MMP9 levels or activities in the airway may be determined using an immunoassay and a biological test sample. In certain embodiment, the MMP9 levels or activities in the airway may be determined using ELISA in a sputum sample from a subject. In one embodiment, the subject is diagnosed with COPD having two or more exacerbations in a year, reduced lung function, and the increased MMP levels or activities in airway.

In some embodiments, the subject is or was a long-term tobacco smoker. In some embodiments, the subject has been exposed to second-hand smoke. In some embodiments, the subject is or has been exposed to workplace chemical fumes, dust, and/or vapors. In some embodiments, the subject is or has been exposed to air pollution. In some embodiments, the subject has a family history of COPD.

Methods of Detection of MMP9

The present disclosure also contemplates methods of detecting MMP9 in a subject, e.g., to detect tissue or fluid or other biological sample associated with a COPD. Thus, methods of diagnosing, monitoring, staging or detecting MMP9 activity in a sample from a patient having COPD are provided.

Samples (e.g., test biological samples) from a subject (e.g., an individual suspected of having or known to have COPD, can be analyzed for MMP9 presence, absence, expression, and/or levels. Also, samples from a subject who is suspected of having COPD may be analyzed for MMP9 presence, absence, expression, and/or levels. For example, such samples can be collected and analyzed by detecting the presence or absence of binding of an MMP9 binding protein, such as an antibody or fragment as described herein, to substance (e.g., protein) in the sample. In some examples, the methods further include comparing the amount of binding detected to an amount of binding to a control sample, or comparing the detected level of MMP9 to a control level of MMP9. In some cases, the methods indicate the presence, absence, or severity of a disease or condition as described herein.

This analysis can be performed prior to the initiation of treatment using an MMP9 binding protein as described herein, or can be done as part of monitoring of progress of COPD treatment. In some embodiments, provided are methods of treatment, carried out by performing the detection assays and initiating, altering, or discontinuing treatment of the subject, for example, based on the results of the diagnostic assay. Such diagnostic analysis can be performed using any sample, including but not limited to tissue, cells isolated from such tissues, and the like. In some cases, the methods are performed on liquid samples, such as blood, plasma, serum, whole blood, saliva, urine, or semen. Tissue samples include, for example, formalin-fixed or frozen tissue sections.

Any suitable method for detection and analysis of MMP9 can be employed. Various diagnostic assay techniques known in the art can be adapted for such purpose, such as competitive binding assays, direct or indirect sandwich assays and immunoprecipitation assays conducted in either heterogeneous or homogeneous phases.

MMP9 binding proteins for use in detection methods can be labeled with a detectable moiety. The detectable moiety directly or indirectly produces a detectable signal. For example, the detectable moiety can be any of those described herein such as, for example, a radioisotope, such as 3H, 14C, 32P, 35S, or 125I, a fluorescent or chemiluminescent compound, such as fluorescein isothiocyanate (FITC), Texas red, cyanin, photocyan, rhodamine, or luciferin, or an enzyme, such as alkaline phosphatase, β-galactosidase or horseradish peroxidase.

Detection can be accomplished by contacting a sample under conditions suitable for MMP9 binding protein binding to MMP9, and assessing the presence (e.g., level) or absence of MMP9 binding protein-MMP9 complexes. A level of MMP9 in the sample in comparison with a level of a reference sample can indicate the presence of COPD-associated tissues having MMP9 activity. The reference sample can be a sample taken from the subject at an earlier time point or a sample from another individual.

In some aspects, MMP9 mRNA is detected, such as by hybridization, such as by chromogenic in situ hybridization (CISH). In some aspects, such detection methods are used when high levels of inflammatory cell-derived MMP9 obscure signal in a desired cell type by other detection method, e.g., by IHC.

Various aspects of the invention are further described and illustrated by way of the several examples which follow, none of which are intended to limit the scope of the invention.

### EXAMPLES

### Example 1

### Evaluation of AB0045 in a Phase I Study

This study is a Phase I double-blind, randomized placebo-controlled multicenter study. The primary objective of this study is to evaluate the safety, tolerability and pharmacokinetics (PK) of AB0045 (i.e., an antibody that binds to MMP9) in subjects with chronic obstructive pulmonary disease (COPD) as assessed by adverse events (AEs) and laboratory abnormalities from baseline to Day 29 plus 30 days. The primary outcome measure is the incidence of adverse events, change from screening in laboratory tests and vital signs, and development of immunogenicity after dosing. This composite endpoint measures the safety and tolerability profile of AB0045. The secondary outcome measures are the PK parameters of AB0045 as measured by AUC, the total amount of drug absorbed by the body by comparing plasma concentration over time, and Cmax, the maximum concentration of drug, from baseline to Day 29. The following PK parameters of AB0045 are measured: Tmax (i.e., the time of Cmax), Clast (i.e., the last observable concentration of drug), Tlast (i.e., the time of Clast), Ctau (i.e., the observed drug concentration at the end of the dosing interval), λz (i.e., the terminal elimination rate constant), CL (i.e., systemic clearance following intravenous administration), and Vz (i.e., apparent volume of distribution following intravenous administration) from baseline to Day 29.

The study has an experimental arm, in which each participant receives 400 mg AB0045 every two weeks for a total of three infusions on Days 1, 15, and 29. The study includes a placebo arm, in which participants receive placebo to match AB0045 every two weeks for a total of three infusions on Days 1, 15, and 29. 400 mg of AB0045 is administered intravenously. The placebo to match AB0045 is also administered intravenously.

The participants are between 40 and 75 years old. Males or non-pregnant, non-lactating females are eligible for the study. Male subjects and female subjects of childbearing potential who engage in heterosexual intercourse must agree to use protocol specified method(s) of contraception. Male subjects must refrain from sperm donation for 90 days post last infusion of the study drug. Inclusion criteria include: a) weight≧45 kg to <120 kg at screening; b) diagnosis of COPD per Global Initiative for Chronic Obstructive Lung Disease (GOLD) guidelines for at least 6 months prior to screening (see Global Initiative for Chronic Obstructive Lung Disease (GOLD). Global Strategy for the Diagnosis, Management, and Prevention of Chronic Obstructive Pulmonary Disease. Updated 2013. Available on the world wide web at -goldcopd.org/guidelines-global-strategy-for-diagnosis-management.html) and anticipated to remain on stable therapy for the duration of the study; c) post-bronchodilator forced expiratory volume in one second (FEV1)≧40% predicted (and, in some embodiments, ≦80% predicted); d) no changes in COPD medications within 30 days prior to randomization; e) hepatic panel [aspartate aminotransferase (AST), alanine aminotransferase (ALT), total bilirubin, direct bilirubin, alkaline phosphatase, lactate dehydrogenase (LDH)]≦2 times the upper limit of the normal range (ULN); f) serum creatinine≦2.0; g) hemoglobin≧8.5 g/dL (both males and females; h) absolute neutrophil count (ANC)≧1.5×10̂9/L (1,500 mm̂3); and i) platelets≧100×10̂9/L.

Exclusion criteria include: a) clinically significant active infection as judged by the investigator during screening; b) known history of HIV, hepatitis B, or hepatitis C during screening (subjects who are hepatitis B surface antigen positive, but who receive a successful series of hepatitis B vaccinations and never had the disease remain eligible); c) a positive QuantiFERON-TB GOLD test during screening; d) a history of malignancy within the last 5 years except for patients who have been treated locally for non-melanoma skin cancer or certain carcinoma in situ; e) any serious cardiac event such as myocardial infarction, unstable or life-threatening arrhythmia, hospitalization for cardiac failure within 6 months prior or randomization or any significant or new electrocardiogram (ECG) finding at Visit 1 as judged by the investigator; f) hospitalization for a respiratory event such as, but not limited to, COPD, pneumonia, bronchiolitis, within the previous 6 months prior to randomization; g) chronic lung disease other than COPD, such as asthma, cystic fibrosis or fibrotic disease, α-1 antitrypsin deficiency, interstitial lung disease, pulmonary thromboembolic disease, or bronchiectasis; g) chronic use of systemic corticosteroids and/or treatment with systemic corticosteroids for an acute exacerbation of COPD (AECOPD) event, or other medical condition not requiring hospitalization, within 90 days of randomization; h) treatment with antibiotics for an AECOPD event, or other medical condition not requiring hospitalization within 90 days of randomization, or any minor medical event not requiring hospitalization within 14 days of randomization; i) treatment with any marketed or investigational biologic within 5 half-lives of the molecule or if unknown within 90 days of screening; and j) subjects currently on nonbiologic immune modulator medications such as: azathioprine, cyclosporine, hydroxychloroquine, leflunomide, methotrexate, mycophenolate mofetil, sulfasalazine, tofacitinib, within 90 days of randomization.

What is claimed is:

1. A method for treating or preventing chronic obstructive pulmonary disease (COPD) in a subject, comprising administering to the subject an effective amount of an anti-Matrix Metalloproteinase 9 (MMP9) antibody or antigen binding fragment thereof.

2. The method of claim 1, wherein the anti-MMP9 antibody or antigen binding fragment thereof binds to an epitope of MMP9, wherein the epitope comprises amino acid residues 104-119, residues 159-166, or residues 191-202 of SEQ ID NO: 27.

3. The method of claim 2, wherein the epitope comprises E111, D113, R162, or I198 of SEQ ID NO: 27.

4. The method of claim 1, wherein anti-MMP9 antibody or antigen binding fragment thereof competes for binding to MMP9 with a protein, wherein the protein binds to amino acid residues 104-119, residues 159-166, or residues 191-202 of SEQ ID NO: 27.

5. The method of claim 4, wherein the protein is an antibody having at least about 95%, 96%, 97%, 98%, 99% or greater identity to the amino acid sequences selected from the group consisting of SEQ ID NOs: 7, 12, 13, 14, 15, 16, 17, and 18.

6. The method of claim 1, wherein the anti-MMP9 antibody or antigen binding fragment thereof comprises a heavy chain variable (VH) region comprising a complementarity-determining region (CDR) having an amino acid sequence selected from the group consisting of SEQ ID NOs: 13, 14, and 15.

7. The method of claim 1, wherein the anti-MMP9 antibody or antigen binding fragment thereof comprises a light chain variable (VL) region comprising a CDR having an amino acid sequence selected from the group consisting of SEQ ID NOs: 16, 17, and 18.

8. The method of claim 1, wherein the anti-MMP9 antibody or antigen binding fragment thereof comprises the VH region comprising the amino acid sequence selected from the group consisting of SEQ ID NOs: 3, 5, 6, 7, and 8.

9. The method of claim 1, wherein the anti-MMP9 antibody or antigen binding fragment thereof comprises the VL region comprising the amino acid sequence selected from the group consisting of SEQ ID NOs: 4, 9, 10, 11, and 12.

10. The method of claim 1, wherein the anti-MMP9 antibody or antigen binding fragment thereof comprises a VH region comprising the amino acid sequence set forth in SEQ ID NO: 7 and a VL region comprising the amino acid sequence set forth in SEQ ID NO: 12.

11. The method of claim 1, wherein the anti-MMP9 antibody or antigen binding fragment thereof comprises a heavy chain comprising the amino acid sequence set forth in SEQ ID NO: 56 and a light chain comprising the amino acid sequence set forth in SEQ ID NO: 57.

12. The method of claim 1, wherein the anti-MMP9 antibody or antigen binding fragment thereof is humanized, chimeric, or human.

13. The method of claim 1, wherein the anti-MMP9 antibody or antigen binding fragment thereof inhibits the enzymatic activity of MMP9.

14. The method of claim 13, wherein the inhibition is non-competitive.

15. The method of claim 1, wherein the anti-MMP9 antibody or antigen binding fragment thereof is administered at a dose of about 100 mg, 150 mg, 200 mg, 300 mg, or 400 mg.

16. The method of claim 1, wherein the anti-MMP9 antibody or antigen binding fragment thereof is administered once every week, once every two weeks, or once every three weeks.

17. The method of claim 1, wherein the anti-MMP9 antibody or antigen binding fragment thereof is administered intravenously, intradermally, or subcutaneously.

18. The method of claim 1, wherein the anti-MMP9 antibody or antigen binding fragment thereof is administered concurrently or sequentially with the one or more therapeutic agents for treating COPD.

19. The method of claim 18, wherein the one or more therapeutic agents for treating COPD is selected from the group consisting of short-acting β2 agonists, short-acting anticholinergics, long-acting β-2 agonists, long-acting anticholinergics, and a combination thereof.

20. The method of claim 19, further comprising β-2 agonist, anticholinergics, inhaled corticosteroids, systemic corticosteroids, methylxanthines, phosphosdiesterase-4 inhibitors, or the combination thereof.
